(19) United States
(12) Patent Application Publication
(10) Pub. No.: US 2007/0036263 A1
(43) Pub. Date: Feb. 15, 2007

(54) Radiation CT method and X-ray CT apparatus

(75) Inventors: Akihiko Nishide, Tokyo (JP); Akira Hagiwara, Tokyo (JP); Kotoko Morikawa, Tokyo (JP); Masatake Nukui, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/500,634

(22) Filed: Aug. 8, 2006

(30) Foreign Application Priority Data
Aug. 9, 2005 (JP) 2005-231062

(51) Int. Cl. H05G 1/60; A61B 6/00; G01N 23/00; G21K 1/12

(52) U.S. Cl. 378/4

(57) ABSTRACT

The present invention is intended to improve the quality of a tomographic image to be produced by an X-ray CT apparatus including an X-ray area detector represented by a multi-array X-ray detector or a flat-panel detector. A conventional (axial) or cine scan is performed on the first and second scanned positions in the direction of a z axis. Projection data items produced by scanning the first scanned position and projection data items produced by scanning the second scanned position are used to reconstruct a tomographic image.

### CROSS REFERENCE TO RELATED APPLICATIONS

This application claims the benefit of Japanese Application No. 2005-231062 filed Aug. 9, 2005.

### BACKGROUND OF THE INVENTION

The present invention relates to a radiation computed tomography (CT) method and an X-ray CT apparatus. More particularly, the present invention is concerned with a radiation CT method for improving the quality of a tomographic image which an X-ray CT apparatus including an X-ray area detector that has a matrix structure and is represented by a multi-array X-ray detector or a flat-panel X-ray detector produces by continuously performing a conventional (axial) scan or a cine scan on different scanned positions on a subject in the direction of the body axis of the subject (the direction of a z axis), and the X-ray CT apparatus.

An X-ray CT apparatus adopting X-rays as a radiation is well-known as an example of a radiation CT apparatus. Known as the X-ray CT apparatus is a type of X-ray CT apparatus including an X-ray area detector that has detector elements arrayed two-dimensionally in the form of a matrix and that is represented by a multi-array X-ray detector or a flat-panel X-ray detector. The multi-array X-ray detector including a plurality of arrays of detector elements is one type of X-ray area detector. The multi-array X-ray detector has a matrix structure in which the detector arrays are juxtaposed in the direction of a z axis corresponding to the direction of a subject's body axis and channels are juxtaposed in a direction parallel to an xy plane.

In general, the X-ray CT apparatus including an X-ray area detector that has a matrix structure and is represented by a multi-array X-ray detector or a flat-panel X-ray detector adopts as an image reconstruction method a cone-beam back projection method represented by a Feldkamp technique or a three-dimensional reconstruction method (refer to, for example, Patent Document 1).

[Patent Document 1] Japanese Unexamined Patent Application Publication No. 2002-336239

When an X-ray CT apparatus including an X-ray area detector adopts a three-dimensional image reconstruction method, an X-ray generator radiates conical X-rays while being located on an xy plane that is orthogonal to a z axis and that contains a field of view. The X-ray area detector detects X-rays transmitted by a subject. The X-ray generator and X-ray area detector rotate about the z axis by one turn, whereby the subject's region to be examined is scanned. After one scan is completed, a data acquisition system composed of the X-ray generator and X-ray area detector and the subject lying down on a cradle are moved from each other by a predetermined distance in the z-axis direction corresponding to the longitudinal direction of a radiographic table. This scanning technique is called conventional (axial) scanning. Since the X-ray area detector has a plurality of arrays of X-ray detector elements juxtaposed in the z-axis direction, a plurality of tomographic images of a subject can be produced by performing one scan.

Moreover, a scanning technique of performing a plurality of conventional scans with an X-ray generator aligned with the same position on the z axis is called cine scanning. A tomographic image expressing a subject's section located at the same position on the z axis is acquired time-sequentially, whereby a time-varying change in the state of the section can be visualized.

When the conventional scanning or cine scanning is adopted in combination with a conventional two-dimensional image reconstruction method, if a tomographic image expressing a subject's section located at a position on a z axis is reconstructed, only projection data items produced by one detector array that is included in an X-ray detector and that is aligned with the position on the z axis are used for image reconstruction.

However, when it comes to a three-dimensional image reconstruction method, if a tomographic image expressing a subject's section located at a position on a z axis is reconstructed, not only projection data items produced by one detector array that is included in an X-ray area detector and that is aligned with the position on the z axis but also projection data items of an X-ray beam having passed through pixel points in the subject's section are employed. Namely, projection data items of X-rays detected by other detector arrays are also utilized. Since projection data items of X-rays having passed through the pixel points are employed in image reconstruction, a tomographic image that is little affected by artifacts and enjoys improved quality can be produced. In particular, when conventional scanning or cine scanning is adopted, three-dimensional image reconstruction would prove effective in reducing artifacts in a tomographic image based on data items detected by a detector array located at an end of the X-ray area detector in the z-axis direction.

However, when the conventional scanning or cine scanning is adopted, if data items detected by a detector array located at a z-coordinate position or a position in the direction of a z axis is used to reconstruct a tomographic image expressing a subject's section aligned with a detector array serving as an edge in the z-axis direction, the tomographic image data has a small number of pixels produced from the projection data items of an X-ray beam having actually passed through the pixel points in the subject's section. In this case, extrapolated projection date items or projection data items of an X-ray beam having passed through adjoining pixel points are substituted for the projection data items of an X-ray beam having actually passed through the pixel points in the subject's section. Even this technique cannot accurately correct an X-ray beam and produce missing data. Therefore, a tomographic image that expresses a subject's section located at a position on the z axis at which the detector array serving as an edge is located is affected by more artifacts than a tomographic image that expresses a subject's section located at a position on the z axis at which a center detector array is located is. Improvement in image quality has been requested.

According to a conventional three-dimensional image reconstruction method, when the conventional scanning or cine scanning is adopted, projection data items detected during one scan are used to reconstruct a tomographic image. For example, assume that projection data items detected at a first position, which serves as an edge in the z-axis direction, during one scan, are used to produce a tomographic image, and that projection data items detected at a second position, which is separated from the first position by a distance corresponding to the width D of an X-ray beam on the z axis that is a center axis of rotation, during the next scan that is a conventional scan or a cine scan are used to produce a tomographic image. In this case, the projection data items detected at the position serving as an edge are not related to the other projection data items when they are used to reconstruct the tomographic image. Therefore, the tomographic images produced during the two scans are less continuous. When multi planar reformation (MPR) based on plane conversion that employs a plurality of tomographic images expressing sections which are successively juxtaposed in the z-axis direction is adopted, streaky or band-like artifacts appear in a position in a three-dimensional image which corresponds to the position of the boundary between the images produced during first and second scans. Thus, image quality is adversely affected by the discontinuity and the three-dimensional image is insufficiently smooth in the z-axis direction.

### SUMMARY OF THE INVENTION

Therefore, an object of the present invention is to provide a radiation CT method for improving the quality of a tomographic image which an X-ray CT apparatus including an X-ray area detector that has a matrix structure and that is represented by a multi-array X-ray detector or a flat-panel X-ray detector produces by continuously performing a conventional (axial) scan or a cine scan on different scanned positions on a subject in the direction of the body axis of the subject (direction of a z axis), and the X-ray CT apparatus.

According to the first aspect of the present invention, there is provided a radiation CT method including: a scan step at which while a radiation generator and a radiation area detector having a matrix structure, being represented by a multi-array radiation detector or a flat panel detector, and being opposed to the radiation generator are rotated with a center axis of rotation, which is located between the radiation generator and radiation area detector, as a center, projection data items of a subject lying down between the radiation generator and radiation area detector are detected; a z-coordinate positional information acquisition step at which z-coordinate positional information concerning a position at which each set of projection data items is detected is acquired on the assumption that the direction of the center axis of rotation corresponds to the direction of a z axis; a three-dimensional image reconstruction step at which a three-dimensional tomographic image is reconstructed based on the detected projection data items in consideration of the z-coordinate positional information; and a tomographic image display step at which the tomographic image is displayed. The radiation CT method further includes: a first scan step of detecting the first projection data items by performing the first conventional (axial) scan or cine scan on the first scanned position in the z-axis direction on a subject; a second scan step of detecting the second projection data items by performing the second conventional scan or cine scan on the second scanned position in the z-axis direction on the subject at which the range in the z-axis direction of a radiation beam substantially communicates with or overlaps the range in the z-axis direction of a radiation beam incident on the first scanned position; and a tomographic image reconstruction step of reconstructing a tomographic image, which expresses a position on the subject that falls within a range from the z-coordinate position at which the first projection data items are detected to the z-coordinate position at which the second projection data items are detected, by utilizing both the first and second sets of projection data items.

In the radiation CT method according to the first aspect, the first conventional (axial) scan or cine scan is performed on the first scanned position on a subject for data acquisition. The second conventional (axial) scan or cine scan is performed on a position on the subject, at which an incident radiation beam adjoins in the direction of a z axis or overlaps a radiation beam incident on the first scanned position, for the purpose of data acquisition. Both the projection data items detected during the first scan and those detected during the second scan are used to reconstruct a tomographic image. Consequently, a portion in a tomographic image expressing a position on the subject located at or near the boundary between the radiation beams incident on the first and second scanned positions respectively or located at or near a position at which the radiation beams overlap is little affected by artifacts. Thus, a high-quality tomographic image is produced.

According to the second aspect of the present invention, there is provided a radiation CT method identical to the radiation CT method according to the first aspect except that: at the tomographic image reconstruction step, projection data items of a radiation beam having passed through respective pixel points in a subject's section are sampled from the first projection data items and the second projection data items; the sets of projection data items sampled from both the first projection data items and second projection data items are weighted and summated; a three-dimensional tomographic image is reconstructed based on the summated projection data items.

In the radiation CT method according to the second aspect, the projection data items detected during the first scan and the projection data items detected during the second scan are weighted and summated in order to produce one set of projection data items. A three-dimensional tomographic image is reconstructed based on the projection data items. Consequently, a portion of the tomographic image expressing a position on the subject located at or near a position on the subject at which radiation beams incident on the first and second scanned positions respectively border on each other or overlap is little affected by artifacts. Thus, a high-quality tomographic image is produced.

According to the third aspect of the present invention, there is provided a radiation CT method including: a scan step at which while a radiation generator and a radiation area detector having a matrix structure, being represented by a multi-array radiation detector or a flat-panel detector, and being opposed to the radiation generator are rotated about a center axis of radiation, which is located between the radiation generator and radiation area detector, as a center, projection data items of a subject lying down between the radiation generator and radiation area detector are detected; a z-coordinate positional information acquisition step at which z-coordinate positional information concerning a position at which each set of projection data items is detected is acquired on the assumption that the direction of the center axis of rotation corresponds to the direction of a z axis; a three-dimensional image reconstruction step at which a three-dimensional tomographic image is reconstructed based on the detected projection data items in consideration of the z-coordinate positional information; and a tomographic image display step at which the tomographic image is displayed. The radiation CT method further includes: a first scan step of detecting the first projection data items by performing the first conventional (axial) scan or cine scan on the first scanned position in the z-axis direction on the subject; a second scan step of detecting the second projection data items by performing the second conventional scan or cine scan on the second scanned position in the z-axis direction on the subject at which the range in the z-axis direction of a radiation beam substantially communicates with or overlaps the range in the z-axis direction of a radiation beam incident on the first scanned position; and a tomographic image reconstruction step of selecting projection data items, which are provided by a radiation beam having passed through pixel points in a subject's section, from each of the first projection data items and second projection data items, weighting and summating the sets of projection data items selected from the first projection data items and second projection data items respectively, and reconstructing a three-dimensional tomographic image on the basis of at least one of the projection data items selected from the first projection data items, the projection data items selected from the second projection data items, and the summated projection data items.

In the radiation CT method according to the third aspect, the first conventional (axial) scan or cine scan is performed on the first scanned position on a subject for the purpose of data acquisition. The second conventional (axial) scan or cine scan is performed on a position on the subject, at which an incident radiation beam adjoins in the z-axis direction or overlaps a radiation beam incident on the first scanned position, for the purpose of data acquisition. One of the projection data items detected during the first scan and the projection data items detected during the second scan is weighted in order to reconstruct a three-dimensional tomographic image, or both of the projection data items detected during the first scan and the projection data items detected during the second scan are weighted and summated in order to reconstruct a three-dimensional tomographic image. Consequently, a portion of a field of view whose projection data items are missing is limited. Eventually, a high-quality tomographic image little affected by artifacts can be produced.

According to the fourth aspect of the present invention, there is provided a radiation CT method including: a scan step at which while a radiation generator and a radiation area detector having a matrix structure, being represented by a multi-array radiation detector or a flat-panel detector, and being opposed to the radiation generator are rotated about a center axis of radiation, which is located between the radiation generator and radiation area detector, as a center, projection data items of a subject lying down between the radiation generator and radiation area detector are detected; a z-coordinate positional information acquisition step at which z-coordinate positional information concerning a position at which each set of projection data items is detected is acquired on the assumption that the direction of the center axis of rotation corresponds to the direction of a z axis; a three-dimensional image reconstruction step at which a three-dimensional tomographic image is reconstructed based on the detected projection data items in consideration of the z-coordinate positional information; and a tomographic image display step at which the tomographic image is displayed. The radiation CT method further includes: an n-th scan step of detecting the n-th projection data items by performing the n-th conventional (axial) scan or cine scan on the n-th scanned position in the z-axis direction on a subject (where n denotes an integer ranging from 1 to N where N denotes an integer equal to or larger than 2) at which the range in the z-axis direction of a radiation beam substantially communicates with or overlaps the range in the z-axis direction of a radiation beam incident on an adjoining scanned position; and a tomographic image reconstruction step of reconstructing a tomographic image, which expresses a position on the subject falling within the range from the z-coordinate position at which the first projection data items are detected to the z-coordinate position at which the N-th projection data items are detected, by utilizing one or a plurality of the first to N-th sets of projection data items.

In the radiation CT method according to the fourth aspect, a conventional (axial) scan or cine scan is performed on two or more different scanned positions in the z-axis direction on a subject in order to acquire projection data items. One or a plurality of the sets of projection data items are used to produce a tomographic image. Consequently, a portion of a tomographic image expressing a position on the subject located at or near the position at which radiation beams incident on a plurality of scanned positions border on each other or overlap is little affected by artifacts. Eventually, a high-quality tomographic image is produced.

According to the fifth aspect of the present invention, there is provided a radiation CT method identical to the radiation CT method according to any of the first to fourth aspects except that the spacing between adjoining scanned positions is substantially equal to or smaller than the width D of a radiation cone beam on the center axis of rotation.

In the radiation CT method according to the fifth aspect, the spacing between adjoining scanned positions is equal to or smaller than the width D of a radiation cone beam on the center axis of rotation. Consequently, the occurrence of missing projection data items can be avoided.

Incidentally, if missing of some projection data items is permitted, the spacing between scanned positions may be larger than the width D.

According to the sixth aspect of the present invention, there is provided a radiation CT method identical to the radiation CT method according to any of the first to fifth aspects except that at the tomographic image reconstruction step, when a plurality of sets of projection data items is utilized, coefficients dependent on the geometric positions and directions of radiation beams that provide respective sets of projection data items are used to weight the sets of projection data items. The sets of projection data items are then summated. A three-dimensional tomographic image is reconstructed based on at least one of the projection data items selected from the n-th projection data items and the summated projection data items.

In the radiation CT method according to the sixth aspect, coefficients dependent on the geometric positions and directions of radiation beams that provide respective sets of projection data items are used to weight a plurality of sets of projection data items, and the sets of projection data items are then summated. Since the plurality of sets of projection data items are weighted and summated so that artifacts will hardly occur, a high-quality tomographic image little affected by artifacts can be produced.

According to the seventh aspect of the present invention, there is provided a radiation CT method identical to the radiation CT method according to any of the first to sixth aspects except that at the scan step, when projection data items are detected, the radiation generator is disposed at the same view angle for the scans performed on the adjoining scanned positions.

In the radiation CT method according to the seventh aspect, since the view angles at which the radiation generator is disposed for the first and second scans respectively are identical to each other, sets of projection data items detected with the radiation generator disposed at the same view angle are weighted and summated. Consequently, an image can be reconstructed without an increase in the number of views to be used for back projection and occurrence of a blur.

According to the eighth aspect of the present invention, there is provided a radiation CT method identical to the radiation CT apparatus according to any of the first to sixth aspects except that: at the scan step, when projection data items are detected, the radiation generator is not necessarily disposed at the same view angle for the scans performed on adjoining scanned positions; and at the tomographic image reconstruction step, when a plurality of sets of projection data items is utilized, if sets of projection data items detected during the respective scans performed on different scanned positions with the radiation generator disposed at the same view angle cannot be sampled, the sets of projection data items are weighted and summated in consideration of the view angles at which the radiation generator is disposed in order to detect the respective sets of projection data items.

In the radiation CT method according to the eighth aspect, even if the view angles at which the radiation generator is disposed for the first and second scans respectively are not necessarily identical to each other, sets of projection data items are appropriately weighted and summated in consideration of the view angles at which the radiation generator is disposed in order to detect the respective sets of projection data items. Even when back projection is performed, an image can be reconstructed properly. Eventually, a high-quality tomographic image little affected by artifacts can be produced.

According to the ninth aspect of the present invention, there is provided a radiation CT method identical to the radiation CT method according to any of the first to sixth aspects except that: at the scan step, when projection data items are detected, the radiation generator is not necessarily disposed at the same view angle for the scans performed on adjoining scanned positions; and at the tomographic image reconstruction step, when a plurality of sets of projection data items is utilized, if sets of projection data items detected during the respective scans performed on different scanned positions with the radiation generator disposed at the same view angle cannot be sampled, the sets of projection data items detected during one scan performed on one scanned position with the radiation generator disposed at a plurality of view angles are weighted and summated, and the resultant projection data items are regarded as projection data items detected with the radiation generator disposed at the same view angle.

In the radiation CT method according to the ninth aspect, even when the view angles at which the radiation generator is disposed for the first and second scans respectively are not identical to each other, sets of projection data items are appropriately weighted so that the sets of projection data items can be regarded as sets of projection data items detected with the radiation generator disposed at the same view angle, and then summated. Therefore, an image can be accurately reconstructed through back projection. Eventually, a high-quality tomographic image little affected by artifacts can be produced.

According to the tenth aspect of the present invention, there is provided a radiation CT method identical to the radiation CT method according to the first aspect except that: at the tomographic image reconstruction step, projection data items provided by a radiation beam having passed through pixel points in a subject's section are selected from sets of projection data items produced by scanning respective scanned positions, three-dimensional tomographic images expressing the respective scanned positions are reconstructed based on the sets of projection data items selected from the sets of projection data items produced by scanning the respective scanned positions, and the tomographic images expressing the respective scanned positions are weighted and summated in order to produce a tomographic image.

In the radiation CT method according to the tenth aspect, the first three-dimensional tomographic image is reconstructed based on projection data items detected during the first scan, and the second three-dimensional tomographic image expressing the same scanned position in the z-axis direction on the subject as the scanned position expressed by the first tomographic image is reconstructed based on projection data items detected during the second scan. The two tomographic images are weighted and summated in order to produce one tomographic image. Consequently, a portion of a tomographic image expressing a position on the subject located at or near a position at which radiation beams incident on the first and second scanned positions respectively border on each other or overlap is little affected by artifacts. Eventually, a high-quality tomographic image can be produced.

According to the eleventh aspect of the present invention, there is provided a radiation CT method identical to the radiation CT method according to the tenth aspect except that coefficients dependent on geometric conditions including scanned positions corresponding to subject's sections, positions in the z-axis direction of the sections, slice thicknesses of the sections, the positions of pixel points in each of the sections, the position and size of a focal spot in the radiation generator, and the position and size of the radiation area detector are used to weight tomographic images expressing the respective scanned positions, and the resultant tomographic images are summated.

In the radiation CT method according to the eleventh aspect, coefficients dependent on geometric conditions including scanned positions corresponding to subject's sections, positions in the z-axis direction of the sections, slice thicknesses of the sections, positions of pixel points in each of the sections, the position and size of the focal spot in the radiation generator, and the position and size of the radiation area detector are used to weight a plurality of tomographic images, and the resultant tomographic images are summated. Consequently, the tomographic images are weighted so that artifacts in the respective tomographic images will be canceled out, and then summated. Eventually, a high-quality tomographic image little affected by artifacts can be produced.

According to the twelfth aspect of the present invention, there is provided a radiation CT method identical to the radiation CT method according to the fourth aspect except that: at the tomographic image reconstruction step, when a plurality of sets of projection data items is utilized, projection data items provided by a radiation beam having passed through pixel points in a subject's section are selected from sets of projection data items produced by scanning respective scanned positions, three-dimensional tomographic images expressing the respective scanned positions are reconstructed based on the sets of projection data items selected from the sets of projection data items produced by scanning the respective scanned positions, the tomographic images expressing the respective scanned positions are weighted and summated in order to produce a tomographic image.

In the radiation CT method according to the twelfth aspect, a plurality of three-dimensional tomographic images are reconstructed based on sets of projection data items provided by radiation beams incident on different scanned positions. The tomographic images are weighted and summated in order to produce one tomographic image. Consequently, a portion of a tomographic image expressing a position on the subject located at or near a position at which radiation beams incident on a plurality of scanned positions border on each other or overlap is little affected by artifacts. Eventually, a high-quality tomographic image can be produced.

According to the thirteenth aspect of the present invention, there is provided an X-ray CT apparatus including: a scan means for rotating an X-ray generator and an X-ray area detector, which is opposed to the X-ray generator, is represented by a multi-array X-ray detector or a flat-panel detector, and has a matrix structure, with a center axis of rotation, which is located between the X-ray generator and X-ray area detector, as a center so as to acquire projection data items of a subject lying down between the X-ray generator and X-ray area detector; a z-coordinate positional information acquisition means for acquiring z-coordinate positional information concerning a position, at which each set of projection data items is detected, on the assumption that the direction of the center axis of rotation corresponds to the direction of a z axis; a three-dimensional image reconstruction means for reconstructing a three-dimensional tomographic image based on the detected projection data items in consideration of the z-coordinate positional information; and a tomographic image display means for displaying the tomographic image. The X-ray CT apparatus further includes: a first scan means for detecting the first projection data items by performing the first conventional (axial) scan or cine scan on the first scanned position in the z-axis direction on the subject; a second scan means for detecting the second projection data items by performing the second conventional scan or cine scan on the second scanned position in the z-axis direction on the subject at which the range in the z-axis direction of an X-ray beam substantially communicates with or overlaps the range in the z-axis direction of an X-ray beam incident on the first scanned position; and a tomographic image reconstruction means for reconstructing a tomographic image, which expresses a position on the subject falling within a range from the z-coordinate position at which the first projection data items are detected to the z-coordinate position at which the second projection data items are detected, by utilizing both the first projection data items and second projection data items.

In the X-ray CT apparatus according to the thirteenth aspect, the radiation CT method according to the second aspect can be preferably implemented.

According to the fourteenth aspect of the present invention, there is provided an X-ray CT apparatus identical to the X-ray CT apparatus according to the thirteenth aspect except that the tomographic image reconstruction means selects projection data items, which are provided by an X-ray beam having passed through pixel points in a subject's section, from each of the first projection data items and the second projection data items, weights and summates the sets of projection data items selected from the first projection data items and second projection data items respectively, and reconstructs a three-dimensional tomographic image on the basis of the summated projection data items.

In the X-ray CT apparatus according to the fourteenth aspect, the radiation CT method according to the second aspect can be preferably implemented.

According to the fifteenth aspect of the present invention, there is provided an X-ray CT apparatus including: a scan means for rotating an X-ray generator and an X-ray area detector, which is opposed to the X-ray generator, is represented by a multi-array X-ray detector or a flat-panel detector, and has a matrix structure, with a center axis of rotation, which is located between the X-ray generator and X-ray area detector, as a center so as to acquire projection data items of a subject lying down between the X-ray generator and X-ray area detector; a z-coordinate positional information acquisition means for acquiring z-coordinate positional information concerning a position, at which each set of projection data items is detected, on the assumption that the direction of the center axis of rotation corresponds to the direction of a z axis; a three-dimensional image reconstruction means for reconstructing a three-dimensional tomographic image on the basis of the detected projection data items in consideration of the z-coordinate positional information; and a tomographic image display means for displaying the tomographic image. The X-ray CT apparatus further includes: a first scan means for detecting the first projection data items by performing the first conventional (axial) scan or cine scan on the first scanned position in the z-axis direction on the subject; a second scan means for detecting the second projection data items by performing the second conventional scan or cine scan on the second scanned position in the z-axis direction on the subject at which the range in the z-axis direction of an X-ray beam substantially communicates with or overlaps the range in the z-axis direction of an X-ray beam incident on the first scanned position; and a tomographic image reconstruction means for selecting projection data items, which are provided by an X-ray beam having passed through pixel points in a subject's section, from each of the first projection data items and second projection data items, weighting and summating the sets of projection data items selected from the first projection data items and second projection data items respectively, and reconstructing a three-dimensional tomographic image on the basis of at least one of the projection data items selected from the first projection data items, the projection data items selected from the second projection data items, and the summated projection data items.

In the X-ray CT apparatus according to the fifteenth aspect, the radiation CT method according to the third aspect can be preferably implemented.

According to the sixteenth aspect of the present invention, there is provided an X-ray CT apparatus including: a scan means for rotating an X-ray generator and an X-ray area detector, which is opposed to the X-ray generator, is represented by a multi-array X-ray detector or a flat-panel detector, and has a matrix structure, with a center axis of rotation, which is located between the X-ray generator and X-ray area detector, as a center so as to acquire projection data items of a subject lying down between the X-ray generator and X-ray area detector; a z-coordinate positional information acquisition means for acquiring z-coordinate positional information concerning a position, at which each set of projection data items is detected, on the assumption that the direction of the center axis of rotation corresponds to the direction of a z axis; a three-dimensional image reconstruction means for reconstructing a three-dimensional tomographic image on the basis of the detected projection data items in consideration of the z-coordinate positional information; and a tomographic image display means for displaying the tomographic image. The X-ray CT apparatus further includes: an n-th scan means for detecting the n-th projection data items by performing the n-th conventional (axial) scan or cine scan on the n-th scanned position in the z-axis direction on the subject (where n denotes an integer ranging from 1 to N where N denotes an integer equal to or larger than 2) at which the range in the z-axis direction of an X-ray beam substantially communicates with or overlaps the range in the z-axis direction of an X-ray beam incident on an adjoining scanned position; and a tomographic image reconstruction means for reconstructing a tomographic image, which expresses a position on the subject located at a position falling within a range from the z-coordinate position at which the first projection data items are detected to the z-coordinate position at which the N-th projection data items are detected, by utilizing one or a plurality of the first to N-th sets of projection data items.

In the X-ray CT apparatus according to the sixteenth aspect, the radiation CT method according to the fourth aspect can be preferably implemented.

According to the seventeenth aspect of the present invention, there is provided an X-ray CT apparatus identical to the X-ray CT apparatus according to any of the thirteenth to sixteenth aspects except that the spacing between adjoining scanned positions is substantially equal to or smaller than the width D of an X-ray cone beam on the center axis of rotation.

In the X-ray CT apparatus according to the seventeenth aspect, the radiation CT method according to the fifth aspect can be preferably implemented.

According to the eighteenth aspect of the present invention, there is provided an X-ray CT apparatus identical to the X-ray CT apparatus according to any of the thirteenth to seventeenth aspects except that when a plurality of sets of projection data items is utilized, the tomographic image reconstruction means uses coefficients, which depend on the geometric positions and directions of X-ray beams providing sets of projection data items, to weight the plurality of sets of projection data items, summates the resultant sets of projection data items, and reconstructs a three-dimensional tomographic image on the basis of at least one of the projection data items selected from the n-th projection data items and the summated projection data items.

In the X-ray CT apparatus according to the eighteenth aspect, the radiation CT method according to the sixth aspect can be preferably implemented.

According to the nineteenth aspect of the present invention, there is provided an X-ray CT apparatus identical to the X-ray CT apparatus according to any of the thirteenth to eighteenth aspects except that when the scan means detects projection data items, the X-ray generator is disposed at the same view angle for the scans performed on adjoining scanned position.

In the X-ray CT apparatus according to the nineteenth aspect, the radiation CT method according to the seventh aspect can be preferably implemented.

According to the twentieth aspect of the present invention, there is provided an X-ray CT apparatus identical to the X-ray CT apparatus according to any of the thirteenth to eighteenth aspects except that: when the scan means detects projection data items, the X-ray generator is not necessarily disposed as the same view angle for the scans performed on adjoining scanned positions; and when a plurality of sets of projection data items is utilized, if projection data items detected during the respective scans performed on different scanned positions with the X-ray generator disposed at the same view angle cannot be sampled, the tomographic image reconstruction means synthesizes sets of projection data items in consideration of the view angles at which the X-ray generator is disposed in order to detect the sets of projection data items.

In the X-ray CT apparatus according to the twentieth aspect, the radiation CT method according to the eighth aspect can be preferably implemented.

According to the twenty-first aspect of the present invention, there is provided an X-ray CT apparatus identical to the X-ray CT apparatus according to any of the thirteenth to eighteenth aspects except that: when the scan means detects projection data items, the X-ray generator is not necessarily disposed at the same view angle for the scans performed on adjoining scanned positions; and when a plurality of sets of projection data items is utilized, if projection data items detected during the respective scans performed on different scanned positions with the X-ray generator disposed at the same view angle cannot be sampled, the tomographic image reconstruction means synthesizes sets of projection data items detected during one scan performed on one scanned position with the X-ray generator disposed at a plurality of view angles, and regards the resultant projection data items as projection data items detected with the X-ray generator disposed at the same view angle.

In the X-ray CT apparatus according to the twenty-first aspect, the radiation CT method according to the ninth aspect can be preferably implemented.

According to the twenty-second aspect of the present invention, there is provided an X-ray CT apparatus according to the X-ray CT apparatus according to the thirteenth aspect except that the tomographic image reconstruction means selects projection data items, which are provided by an X-ray beam having passed through pixel points in a subject's section, from sets of projection data items provided by X-ray beams incident on respective scanned positions, reconstructs three-dimensional images expressing the scanned positions on the basis of the sets of projection data items selected from the set of projection data items provided by the X-ray beams incident on the respective scanned positions, and weights and summates the resultant tomographic images expressing the scanned positions so as to product a tomographic image.

In the X-ray CT apparatus according to the twenty-second aspect, the radiation CT method according to the tenth aspect can be preferably implemented.

According to the twenty-third aspect of the present invention, there is provided an X-ray CT apparatus identical to the X-ray CT apparatus according to the twenty-second aspect except that coefficients dependent on geometric conditions including scanned positions corresponding to subject's sections, positions in the z-axis direction of the sections, slice thicknesses of the sections, the positions of pixel points in each of the sections, the position and size of the focal spot in the X-ray generator, and the position and size of the X-ray area detector are used to weight tomographic images expressing the respective scanned positions.

In the X-ray CT apparatus according to the twenty-third aspect, the radiation CT method according to the eleventh aspect can be preferably implemented.

According to the twenty-fourth aspect of the present invention, there is provided an X-ray CT apparatus identical to the X-ray CT apparatus according to the sixteenth aspect except that: when a plurality of sets of projection data items is utilized, the tomographic image reconstruction means selects projection data items, which are provided by an X-ray beam having passed through pixel points in a subject's section, from sets of projection data items provided by X-ray beams incident on respective scanned positions, reconstructs three-dimensional tomographic images, which express the respective scanned positions, on the basis of the sets of projection data items selected from the sets of projection data items provided by X-ray beams incident on the respective scanned positions, and weights and summates the tomographic images expressing the scanned positions so as to product a tomographic image.

In the X-ray CT apparatus according to the twenty-fourth aspect, the radiation CT method according to the twelfth aspect can be preferably implemented.

According to a radiation CT method and an X-ray CT apparatus in which the present invention is implemented, even when a field of view is located at an edge of an X-ray beam incident on a certain scanned position, the quality of a tomographic image expressing the field of view can be improved. Furthermore, the quality of a tomographic image expressing any slice thickness, a tomographic image expressing any position in a z-axis direction, a plane-converted image, or a three-dimensional image can be improved.

A CT method and an X-ray CT apparatus in accordance with the present invention can be used to produce a tomographic image of a subject. Moreover, the X-ray CT apparatus can be adapted to an X-ray CT apparatus for medical or industrial purposes or an X-ray CT-PET system or an X-ray CT-SPECT apparatus in which the X-ray CT apparatus is used in combination with any other modality.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing an X-ray CT apparatus in accordance with the first embodiment.

FIG. 2 is an explanatory diagram showing the geometric dispositions of an X-ray tube and a multi-array X-ray detector which are seen in the direction of a z axis.

FIG. 3 is an explanatory diagram showing the geometric dispositions of the X-ray tube and multi-array X-ray detector which are seen in the direction of an x axis.

FIG. 4 is a flowchart outlining actions to be performed in the X-ray CT apparatus in accordance with the first embodiment.

FIG. 5 is an explanatory diagram showing the first scanned position and an X-ray beam.

FIG. 6 is an explanatory diagram showing the geometric dispositions shown in FIG. 5 in a deformed manner.

FIG. 7 is an explanatory diagram showing the second scanned position and an X-ray beam.

FIG. 8 is an explanatory diagram showing the geometric dispositions shown in FIG. 7 in a deformed manner.

FIG. 9 shows an example of a format for projection data items.

FIG. 10 is an explanatory diagram showing coefficients defined by a direction-of-arrays filter.

FIG. 11 is an explanatory diagram showing a slice thickness that is larger in the perimeter of a field of view than in the center thereof.

FIG. 12 is an explanatory diagram showing coefficients defined by a direction-of-arrays filter and varied depending on a channel.

FIG. 13 is an explanatory diagram showing a slice thickness that is uniform even in the center of a field of view and in the perimeter thereof.

FIG. 14 is an explanatory diagram showing coefficients defined by the direction-of-arrays filter and used to reduce a slice thickness.

FIG. 15 is a flowchart describing three-dimensional back projection employed in the first embodiment.

FIGS. 16a and 16b are conceptual diagrams showing projection of lines of pixel points in a field of view P in the direction of X-ray transmission.

FIG. 17 is a conceptual diagram showing the lines of pixel points in the field of view P projected onto a detector surface.

FIGS. 18a and 18b are conceptual diagrams showing X-ray beams that pass through the same pixel points g in the field of view P and that are incident on different scanned positions in the z-axis direction.

FIGS. 19a and 19b are conceptual diagrams showing projection data items D0 representing pixel points in a field of view P and being produced with the X-ray tube disposed at a view angle view=0°.

FIGS. 20a and 20b are conceptual diagrams showing back projection pixel data items D2 representing the pixel points in the field of view P and being produced with the X-ray tube disposed at a view angle view=0°.

FIG. 21 is a conceptual diagram showing synthetic back projection pixel data items D2′ representing the pixel points in the field of view P and being produced with the X-ray tube disposed at a view angle view=0°.

FIGS. 22a and 22b are conceptual diagrams showing a case where view angles at which the X-ray tube is disposed for scans to be performed on the first and second scanned positions respectively are identical to each other.

FIGS. 23a and 23b are conceptual diagrams showing a case where phases of view angles at which the X-ray tube is disposed for scans to be performed on the first and when second scanned positions are different from each other.

FIGS. 24a and 24b are conceptual diagrams showing a case where phases of view angles at which the X-ray tube is disposed for scans to be performed on the first and when second scanned positions are different from each other, and a difference between view angles at which the X-ray tube is disposed for the scan to be performed on the first scanned position disagrees with a difference between view angles at which the X-ray tube is disposed for the scan to be performed on the second scanned position.

FIG. 25 is an explanatory diagram showing the relationship between a distance from a center axis of rotation to a pixel point and an X-ray beam.

FIG. 26 is an explanatory diagram showing production of back projection data items D3 through pixel-by-pixel summation of sets of back projection pixel data items D2′ produced from all views.

FIG. 27 is a conceptual diagram showing a circular field of view P.

FIGS. 28a and 28b are conceptual diagrams showing X-ray beams that have passed through the same pixel point g in the same field of view P and its neighborhood and that are incident on different scanned positions in the z-axis direction.

FIG. 29 is a conceptual diagram showing a case where the spacing between the first and second scanned positions is short.

FIG. 30 is an explanatory diagram showing the dispositions shown in FIG. 29 in a deformed manner.

FIG. 31 is an explanatory diagram showing a plurality of X-ray beams passing through the same pixel point according to the second embodiment.

FIG. 32 is a flowchart outlining actions to be performed in an X-ray CT apparatus in accordance with the third embodiment.

FIG. 33 is a flowchart describing three-dimensional back projection employed in the third embodiment.

FIG. 34 is an explanatory diagram showing a field of view corresponding to each scanned position and a final tomographic image.

FIG. 35 is a flowchart outlining actions to be performed in an X-ray CT apparatus in accordance with the fourth embodiment.

FIG. 36 shows a plurality of fields of view and the first scanned position according to the fourth embodiment.

FIG. 37 shows the plurality of fields of view and the second scanned position according to the fourth embodiment.

FIG. 38 is an explanatory diagram showing weighting coefficients employed in the fourth embodiment.

FIG. 39 is a flowchart describing three-dimensional back projection employed in the fourth embodiment.

### DETAILED DESCRIPTION OF THE INVENTION

The present invention will be described by taking illustrated embodiments for instance. Noted is that the present invention is not limited to the embodiments.

[First Embodiment]

According to the first embodiment, data items produced by summating weighted projection data items produced by scanning different scanned positions are used to reconstruct an image. Thus, image quality is improved.

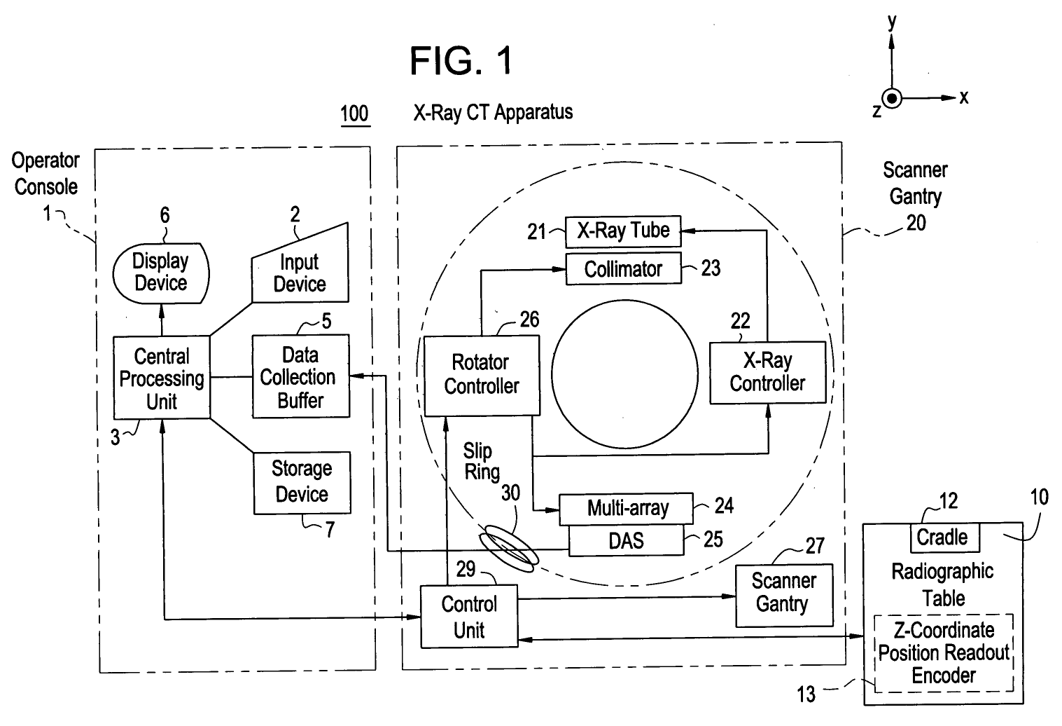

FIG. 1 is a block diagram showing the configuration of an X-ray CT apparatus according to the first embodiment.

The X-ray CT apparatus 100 includes an operator console 1, a radiographic table 10, and a scanner gantry 20.

The operator console 1 includes an input device 2 that receives an operator's entry, a central processing unit 3 that performs preprocessing, image reconstruction, post-processing, and the like, a data collection buffer 5 in which X-ray detector data items acquired by the scanner gantry 20 are collected, a display device 6 on which a tomographic image reconstructed based on projection data items produced by preprocessing the X-ray detector data items is displayed, and a storage device 7 in which programs, X-ray detector data items, projection data items, and tomographic images are stored.

The radiographic table 10 has a cradle 12 on which a subject lies down and which is inserted into or drawn out of the bore of the scanner gantry 20. The cradle 12 is lifted, lowered, or moved rectilinearly with respect to the radiographic table 10 by a motor incorporated in the radiographic table 10. A z-coordinate position on the cradle 12 is accurately detected by a z-coordinate position readout encoder 13 incorporated in the radiographic table 10.

The scanner gantry 20 includes an X-ray tube 21, an X-ray controller 22, a collimator 23, a multi-array X-ray detector 24, a data acquisition system (DAS) 25, a rotator controller 26 that controls the X-ray tube 21 and others which rotate about the body axis of a subject, and a control unit 29 that transfers control signals to or from the operator console 1 and radiographic table 10. A scanner gantry tilt controller 27 allows the scanner gantry 20 to tilt forward or backward at approximately ±30° with respect to the z-axis direction. The projection data items are then analog-to-digital converted by the DAS 25, and transferred to the data collection buffer 5 via a slip ring 30.

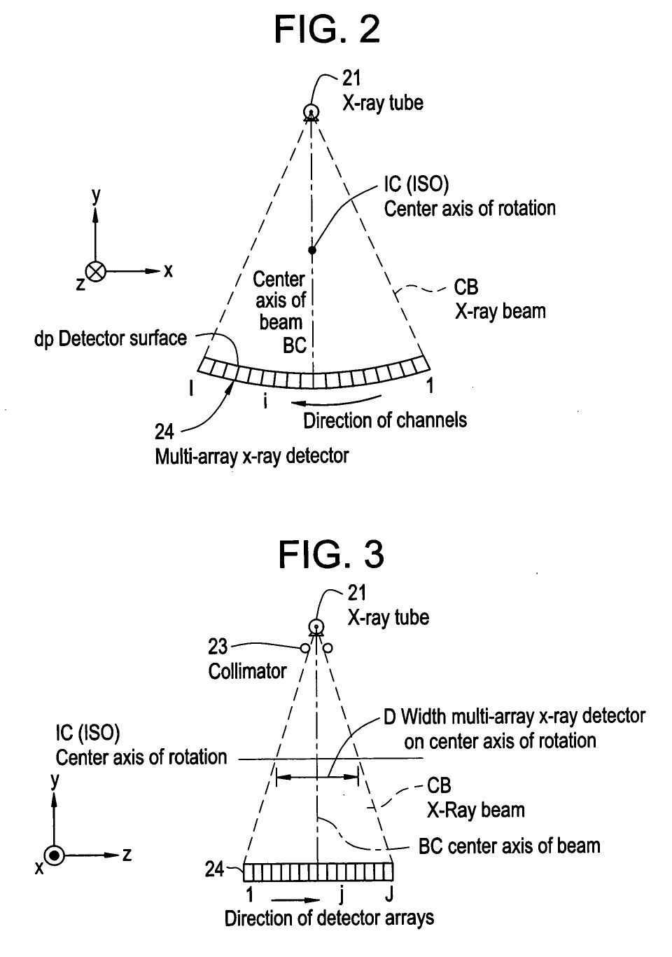

FIG. 2 and FIG. 3 are explanatory diagrams showing the geometric dispositions of the X-ray tube 21 and multi-array X-ray detector 24.

The X-ray tube 21 and multi-array X-ray detector 24 rotate about a center axis of rotation IC. Assuming that a vertical direction is a y-axis direction, a horizontal direction is an x-axis direction, and a table-advancing direction perpendicular to the x and y directions is a z-axis direction, a plane on which the X-ray tube 21 and multi-array X-ray detector 24 rotate is an xy plane. Moreover, a moving direction in which the cradle 12 moves is the z-axis direction.

In conventional (axial) scanning or cine scanning, the X-ray tube 21 and multi-array X-ray detector 24 are rotated about the center axis of rotation IC. When X-ray detector data items are acquired, the cradle 12 is immobilized with the X-ray tube aligned with a scanned position in the z-axis direction.

The X-ray tube 21 generates an X-ray beam that is called a cone beam CB. When the direction of the center axis BC of the cone beam CB is parallel to the y-axis direction, the X-ray tube shall be regarded to be disposed at a view angle of 0°.

The multi-array X-ray detector 24 includes the first to J-th detector arrays where J denotes, for example, 256. Each detector array has the first to I-th channels where I denotes, for example, 1024.

As shown in FIG. 3, D denotes the width in the z-axis direction of the multi-array X-ray detector 24 on the center axis of rotation IC (corresponding to the width in the z-axis direction of an X-ray beam CB on the center axis of rotation IC).

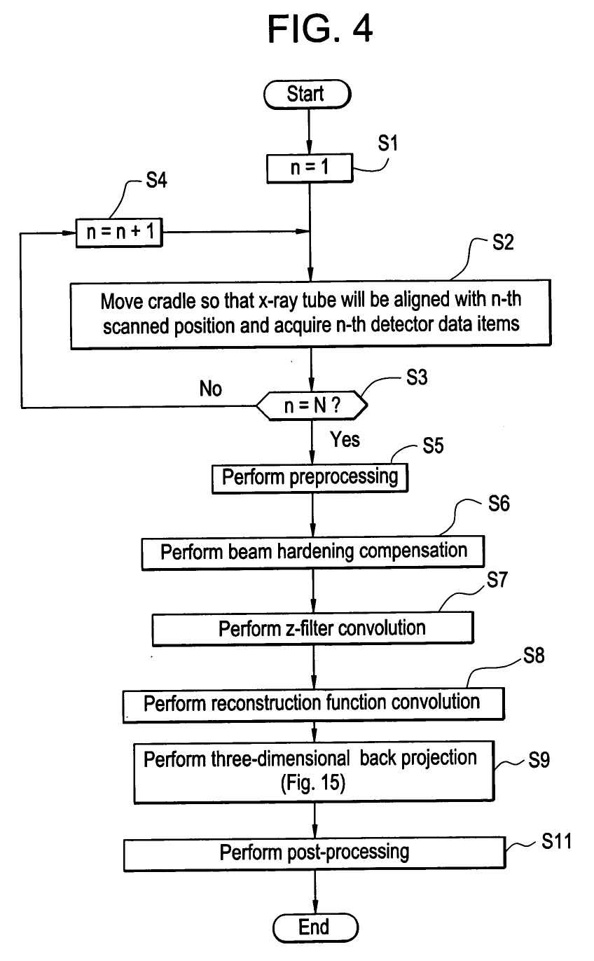

FIG. 4 is a flowchart outlining actions to be performed in the X-ray CT apparatus 100.

At step S1, a scanned position counter n is initialized to 1.

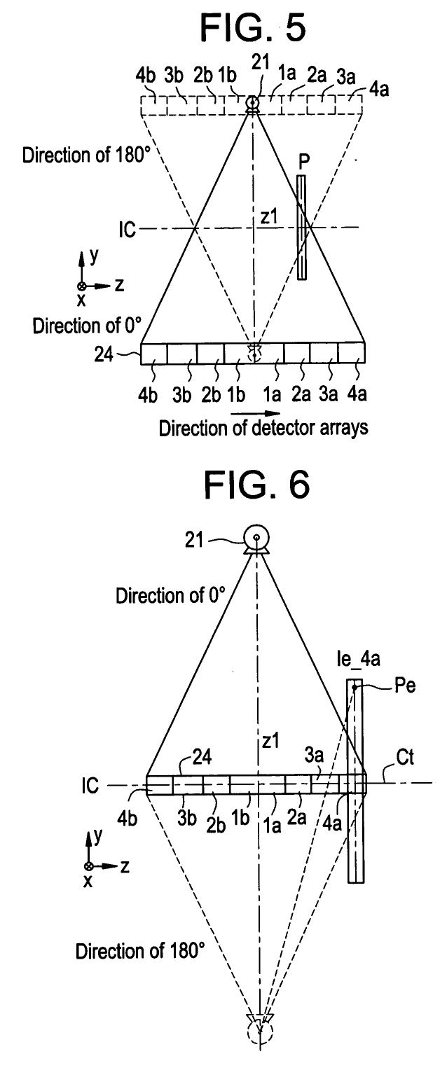

At step S2, the cradle is moved so that the X-ray tube 21 will be aligned with the n-th scanned position zn in the z-axis direction, and the X-ray tube 21 and multi-array X-ray detector 24 are rotated about the center axis of rotation IC. A conventional (axial) scan or cine scan is performed with the cradle 12 immobilized. The n-th X-ray detector data items to which z-axis direction positional information concerning a position in the z-axis direction is appended are acquired. As shown in FIG. 5, the cradle is moved so that the X-ray tube will be aligned with the first scanned position z1 in the z-axis direction. The first X-ray detector data items are then acquired. In FIG. 5, a solid line indicates an X-ray beam irradiated from the X-ray tube disposed at a view angle 0°, and a dot line indicates an X-ray beam irradiated from the X-ray tube disposed at a view angle 180°. P denotes an example of a field of view.

FIG. 6 shows the geometrical dispositions shown in FIG. 5 in a deformed manner as if the multi-array X-ray detector 24 were located on the center axis of rotation IC. The field of view P shown in FIG. 5 is deformed to be a field of view Ie_4a in FIG. 6. A detector element 3a included in the multi-array X-ray detector 24 and located on a segment linking the X-ray tube 21 and a pixel point Pe in the field of view Ie_4a acquires X-ray detector data representing the pixel point Pe. On the other hand, if the segment linking the X-ray tube 21 and the pixel point Pe in the field of view Ie_4a is not drawn or a detector element included in the multi-array X-ray detector 2 is not located on the segment linking the X-ray tube 21 and the pixel point Pe in the field of view Ie_4a, X-ray detector data representing the pixel point Pe is not acquired. For example, referring to FIG. 6, X-ray detector data representing the pixel point Pe cannot be acquired with the X-ray tube disposed at a view angle 0°, but can be acquired by the detector element 3a with the X-ray tube disposed at a view angle 180°.

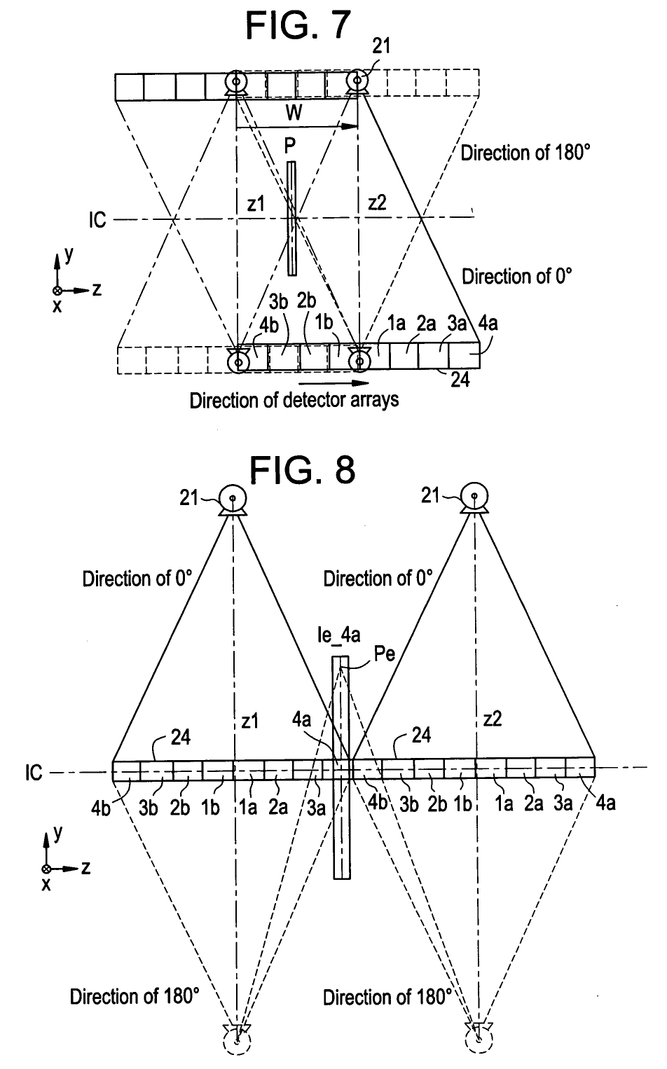

Referring back to FIG. 4, at steps S3 and S4, step S2 is repeated until the count value n becomes equal to the N value (≧2). For example, when the count value n equals 2, the cradle is, as shown in FIG. 7, moved by a distance W in the z-axis direction until the X-ray tube is aligned with the second scanned position z2 therein. The second detector data items are then acquired. Herein, 0<W≦D shall be established. In FIG. 7 the distance W is approximately equal to the width D.

FIG. 8 shows the geometric dispositions shown in FIG. 7 in a deformed manner as if the multi-array X-ray detector 24 were located on the center axis of rotation IC. For example, referring to FIG. 8, as long as the X-ray tube is disposed at a view angle 0°, X-ray detector data representing a pixel point Pe can be acquired from neither the first scanned position z1 nor the second scanned position z2. When the X-ray tube is disposed at a view angle 180°, the X-ray detector data can be acquired from the first scanned position z1 by the detector element 3a, and acquired from the second scanned position z2 by a detector element 4b.

Referring back to FIG. 4, at step S5, preprocessing including offset nulling, logarithmic conversion, X-ray dose correction, and sensitivity correction is performed on X-ray detector data items. This results in projection data items Din(view,j,i) each of which is identified with a view angle view, a detector array number j, and a channel number i.

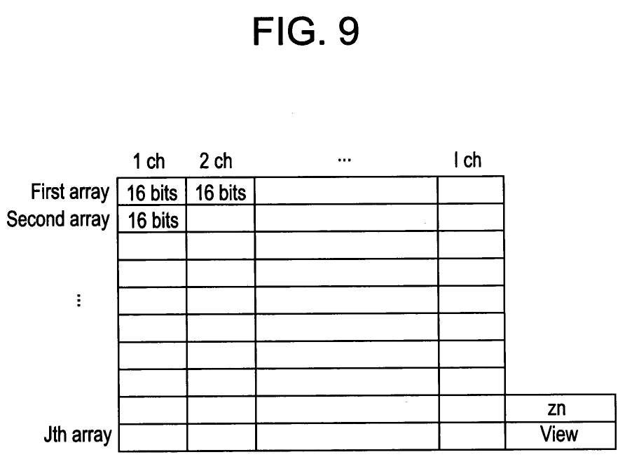

FIG. 9 shows an example of a format for projection data items constituting one view.

The same z-axis direction positional information is appended to the projection data items constituting one view.

At step S6, beam hardening compensation is performed on the projection data items Din(view,j,i). The beam hardening compensation is expressed by, for example, a polynomial expression presented below.
Dout(view,j,i)=(Din(view,j,i)×(B₀(j,i)+B₁(j,i)×Din(view,j,i)+B₂(j,i)×Din(view,j,i)²)
where B₀, B₁, and B₂ denotes beam hardening coefficients.

At this time, the beam hardening compensation is independently performed on data items detected by each channel or on data detected by a detector element belonging to each channel and each detector array of the multi-array X-ray detector 24. Therefore, even if the characteristics of X-rays irradiated from the X-ray tube 21 vary in the z-axis direction, the difference in the characteristic concerning beam hardening of X-rays incident on each detector array from X-rays incident on another detector array can be compensated.

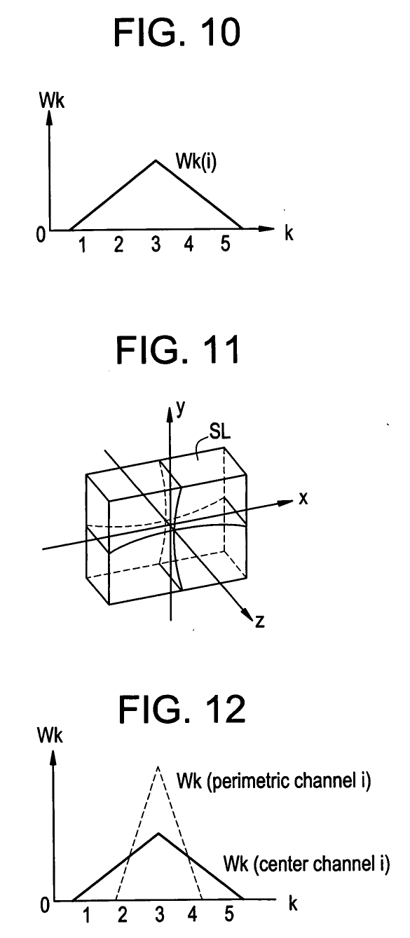

At step S7, z filter convolution is performed in order to filter projection data items Dout(view,j,i), which have undergone the beam hardening compensation, in the z direction (direction of arrays). Specifically, direction-of-arrays filtering coefficients Wk(i) shown in FIG. 10 are applied to the projection data items Dout(view,j,i) in the direction of arrays, whereby projection data items Dcor(view,j,i) are produced.

Projection data items Dout(view,j,i) having undergone z-filter convolution are expressed as follows:

$$Dcor ( view , j , i ) = {\sum }_{k = 1}^{5} ( Dout ( view , j + k - 3 , i ) \times Wk ( i ) )$$

where

$${\sum }_{k = 1}^{5} ( Wk ( i ) ) = 1$$

is established. Incidentally, projection data items Dout(view,J,i) detected by a detector array having the maximum array number J are expressed as follows:
Dout(view,−1,i)=Dout(view,0,i)=Dout(view,1,i)
Dout(view,J,i)=Dout(view,J+1,i)=Dout(view,J+2,i)

When the direction-of-arrays filtering coefficients are varied depending on a channel, a slice thickness can be controlled based on a distance from the center of a field of view.

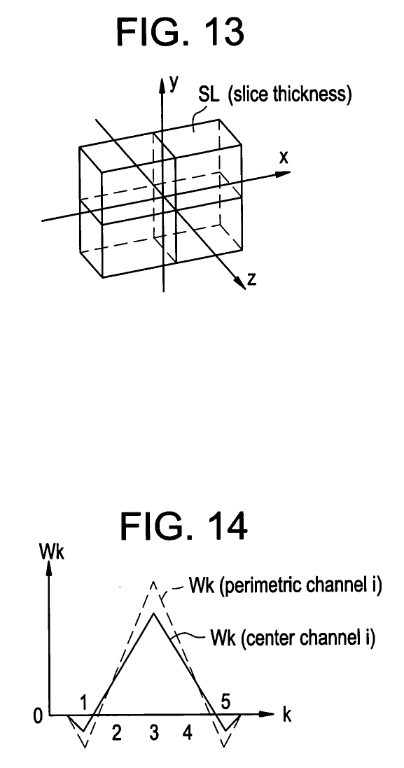

In general, a slice thickness is larger in the perimeter of a field of view than in the center thereof in the same manner as the slice thickness SL shown in FIG. 11. Consequently, as shown in FIG. 12, direction-of-arrays filtering coefficients Wk (center channel i) having a wide variance are applied to data items detected on the center channel, and direction-of-arrays filtering coefficients Wk (perimetric channel i) having a narrow variance are applied to data items detected on a perimetric channel. Thus, as shown in FIG. 13, the slice thickness SL becomes uniform even in the center of the field of view and in the perimeter thereof.

When a slice thickness is slightly increased by adjusting the direction-of-arrays filtering coefficients Wk(i), both artifacts and noises are reduced. Consequently, a degree of reduction in artifacts and a degree of reduction in noises can be controlled. In other words, the quality of a three-dimensional tomographic image can be controlled.

As shown in FIG. 14, when the direction-of-arrays filtering coefficients Wk(i) are determined in order to realize a de-convolution filter, a tomographic image expressing a small slice thickness can be produced.

Referring back to FIG. 4, at step S8, reconstruction fimction convolution is performed. Specifically, data items are Fourier-transformed, applied a reconstruction function, and then inverse-Fourier-transformed. Assuming that Dr(view,j,i) denotes projection data items having undergone the reconstruction function convolution, Kernel(j) denotes the reconstruction function, and * denotes convolution, the reconstruction function convolution is expressed as follows:
Dr(view,j,i)=Dcor(view,j,i)*Kernel(j)

Since the reconstruction function Kernel(j) is independently convoluted to data items detected by each detector array, the difference in the characteristic concerning noises or a resolution of data items detected by each detector array from the characteristic of those detected by another detector array can be compensated.

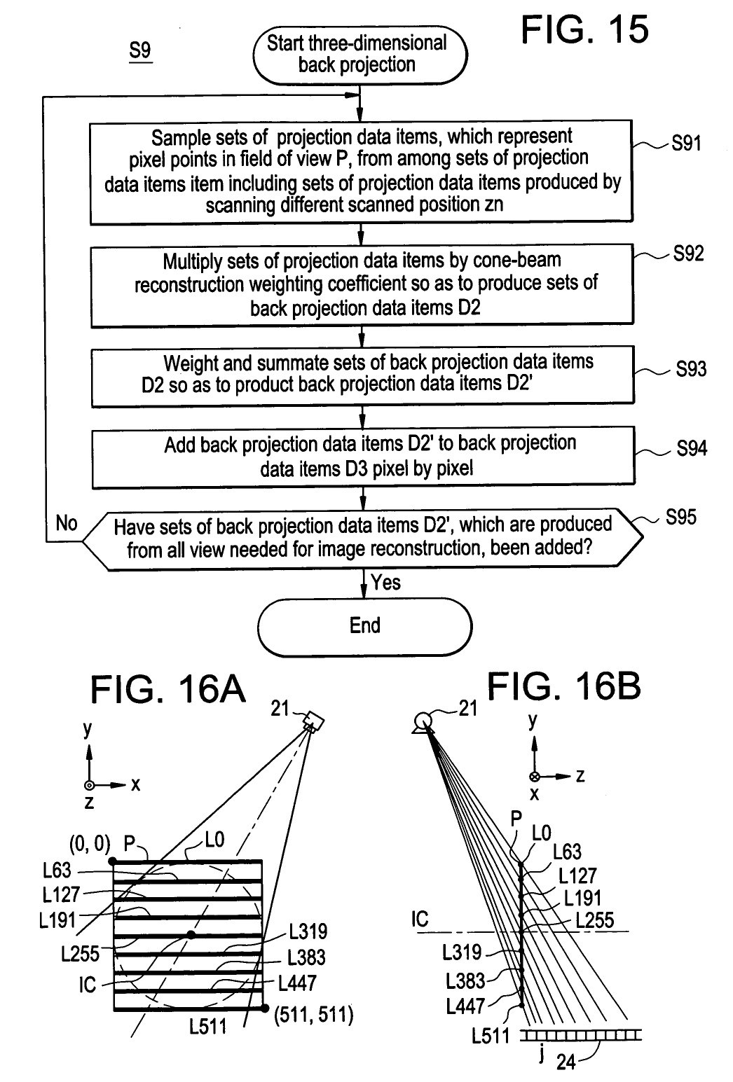

At step S9, three-dimensional back projection is performed on projection data items Dr(view,j,i) in order to produce back projection data items D3(x,y,z), that is, tomographic image data. The three-dimensional back projection will be described later with reference to FIG. 15.

At step S11, post-processing including image filter convolution and CT number transform is performed on back projection data items D3(x,y,z), that is, tomographic image data in order to produce a displayable tomographic image.

Assuming that D4(x,y,z) denotes data items having undergone image filter convolution and Filter(z) denotes an image filter, the image filter convolution is expressed as follows:
D4(x,y,z)=D3(x,y,z)*Filer(z)

Since image filter convolution can be independently performed on data items expressing each slicing position in the z-axis direction on a subject, the difference in the characteristic concerning noises or a resolution of data items expressing each slicing position from the characteristic of those expressing another slicing position can be compensated.

A produced tomographic image is displayed on the display device 6.

FIG. 15 is a flowchart describing three-dimensional back projection (step S9 in FIG. 4).

At step S91, one of all views needed to reconstruct a tomographic image (views detected by rotating the X-ray tube 360° or 180°+ the angle of a fan beam) is focused. Projection data items included in the focused view and representing pixel points in a field of view P are sampled from sets of projection data items including sets of projection data items produced by scanning a different scanned position zn, whereby sets of projection data items D0(view,x,y) are produced.

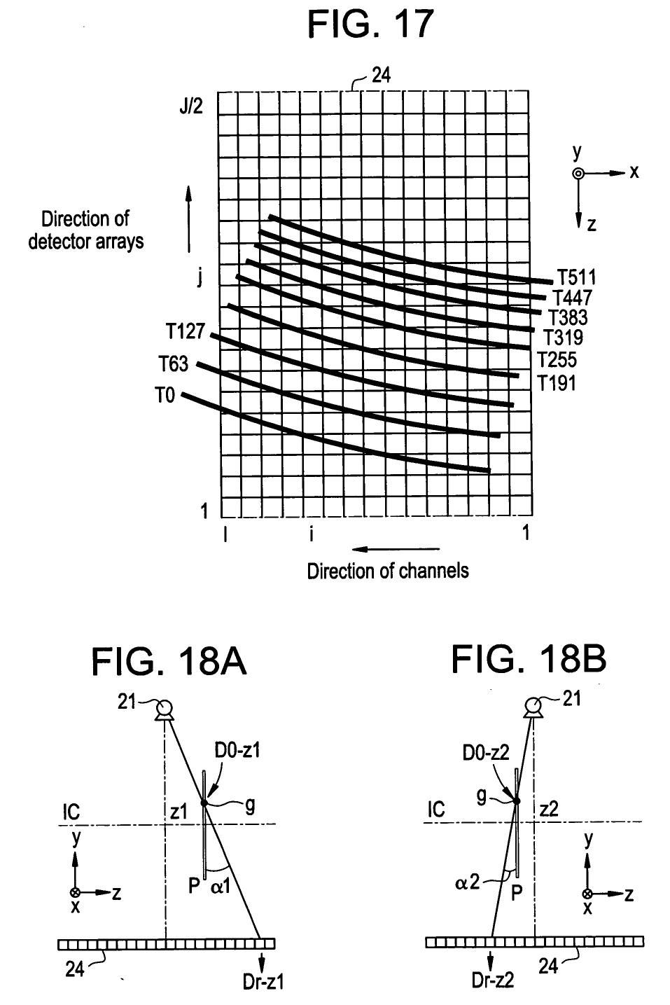

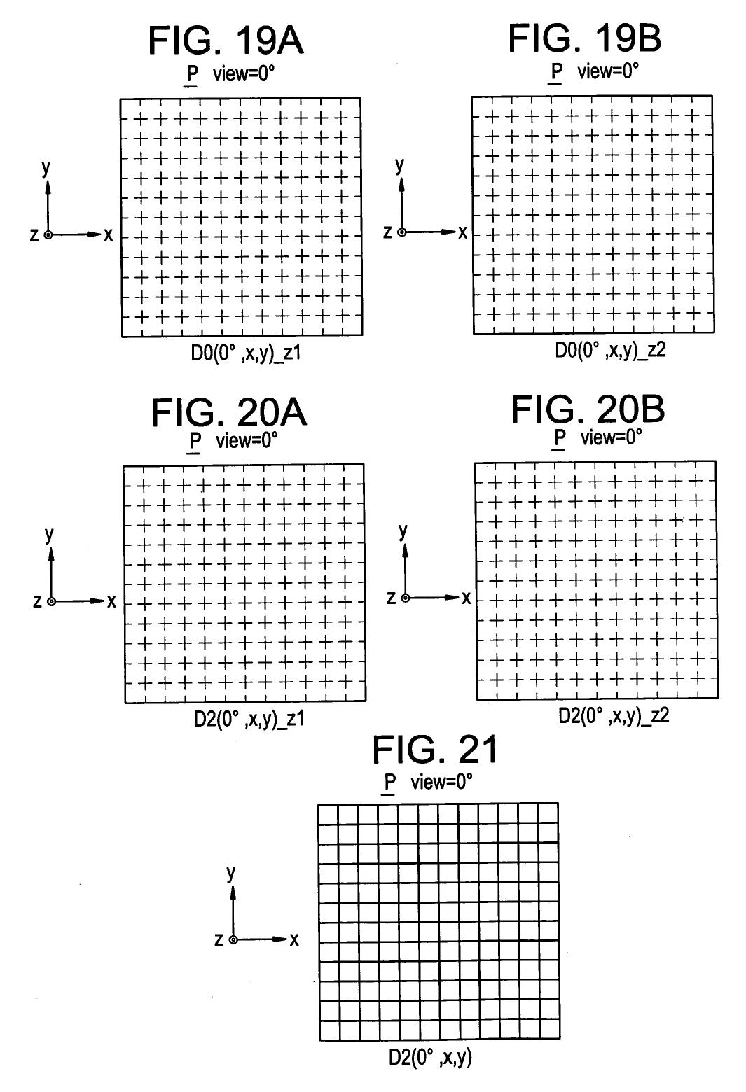

As shown in FIG. 16, a square field of view P has 512 pixel points arrayed in rows and columns in parallel with the xy plane. A line of pixel points L0 parallel to an x axis and indicating a y-coordinate of 0, a line of pixel points L63 indicating a y-coordinate of 63, a line of pixel points L127 indicating a y-coordinate of 127, a line of pixel points L191 indicating a y-coordinate of 191, a line of pixel points L255 indicating a y-coordinate of 255, a line of pixel points L319 indicating a y-coordinate of 319, a line of pixel points L383 indicating a y-coordinate of 383, a line of pixel points L447 indicating a y-coordinate of 447, and a line of pixel points L511 indicating a y-coordinate of 511 are taken for instance. Projection data items detected from lines T0 to T511 formed by projecting the lines of pixel points L0 to L511 on the surface of the multi-array X-ray detector 24 in the direction of X-ray beam transmission are regarded as projection data items Dr(view,x,y) representing the lines of pixel points L0 to L511. Part of a line may come out of the multi-array X-ray detector 24 in the direction of channels in the same manner as, for example, part of the line T0 shown in FIG. 17 does. In this case, projection data items Dr(view,x,y) to be detected from the line are set to 0s. If part of a line comes out in the direction of detector arrays, missing projection data items Dr(view,x,y) are extrapolated. This processing is performed on sets of projection data items produced by scanning a different scanned position in order to produce projection data items D0(view,x,y) representing the lines of pixel points L0 to L511. For example, as shown in FIG. 18 and FIG. 19, sets of projection data items D0_z1 and D0_z2 provided by X-ray beams having passed through pixel points g are produced.

Referring back to FIG. 15, at step S92, projection data items D0 are multiplied by a cone-beam reconstruction weighting coefficient in order to produce sets of back projection data items D2_z1 and D2_z2 as shown in FIG. 20

The cone-beam reconstruction weighting coefficient w(x,y) will be described below.

In the case of fan-bean image reconstruction, assuming that γ denotes an angle at which a segment linking the focal spot in the X-ray tube 21 disposed at a view angle view of βa 9and a pixel point g(x,y) in the field of view P (xy plane) meets the center axis Bc of an X-ray beam, and βb denotes an opposite view angle view, the opposite view angle βb is expressed as follows:
βb=βa+180°−2γ

Assuming that αa and αb denote angles at which an X-ray beam passing through a pixel point g(x,y) in the field of view P and the opposite X-ray beam meet the field of view P, projection data items are multiplied by the cone-beam reconstruction weighting coefficient ωa or ωb dependent on the angle αa or αb in order to produce back projection data items D2(0,x,y).
D2(0,x,y)=ωa·D0(0,x,y)_{—}a+ωb·*D0(0,x,y)_{—}b
where D0(0,x,y)_a denotes projection data items detected with the X-ray tube disposed at the view angle βa, and D0(0,x,y)_b denotes projection data items detected with the X-ray tube disposed at the view angle βb.

Incidentally, the sum of the cone-beam reconstruction weighting coefficients ωa and ωb associated with the X-ray beam and opposite X-ray beam is a unity, that is, ωa+ωb=1 is established.

As mentioned above, projection data items are multiplied by either of the cone-beam reconstruction weighting coefficients ωa and ωb, and the resultant sets of projection data items are summated. This is helpful in reducing cone-angle artifacts.

For example, the cone-beam reconstruction weighting coefficients ωa and ωb may be calculated as described below.

Assuming that ƒ( ) denotes a function and γmax denotes a half of the angle of a fan beam, the following equations are drawn out:
ga=ƒ(γmax,αa,βa)
gb=ƒ(γmax,αb,βb)
xa=2·ga^{q}/(ga^{q}+gb^{q})
xb=2·gb^{q}/(ga^{q}+gb^{q})
ωa=xa²·(3−2xa)
ωb=xb²·(3−2xb)
Herein, for example, q equals 1.

For example, assuming that ƒ( ) denotes a function max[] providing a larger one of 0 and {(π/2+γmax).|βa|}, ga and gb are rewritten as follows:
ga=max[0,{(π/2γmax)−|βa|}]□|tan (αa)|
gb=max[0,{(π/2γmax)−|βb|}]□|tan (αb)|

In the case of fan-beam image reconstruction, the projection data items D0 representing the pixel points in the field of view P are multiplied by a distance coefficient. The distance coefficient is provided as (r1/r0)² where r0 denotes a distance from the focal spot in the X-ray tube 21 to a detector element that belongs to a detector array j and a channel i included in the multi-array X-ray detector 24 and that detects projection data D0, and r1 denotes a distance from the focal spot in the X-ray tube 21 to a pixel point in the field of view P represented by the projection data D0.

In the case of parallel-ray beam image reconstruction, the projection data items D0 representing the pixel points in the field of view P are multiplied by the cone-beam reconstruction weighting coefficient alone.

An X-ray beam and an opposite X-ray beam that pass through a pixel point, which is, for example, a center pixel point Ct in a field of view Ie_{—}4a located as shown in FIG. 6 on the center axis of rotation IC, are contained in an X-ray beam CB incident on one scanned position. In this case, back projection data D2(0,x,y) may be, as mentioned above, produced from the projection data D0(0,x,y)_a and projection data D0(0,x,y)_b.

However, if an X-ray beam and an opposite X-ray beam passing through, for example, a pixel point Pe shown in FIG. 6 are not contained in an X-ray beam CB incident on one scanned position, missing projection data is extrapolated in order to produce back projection data D2(0,x,y). Otherwise, the cone-beam reconstruction weighting coefficient to be applied to imperfect projection data items is set to 0 or unused in order to produce back projection data items including back projection data D2(0,x,y). In this case, degradation in image quality may be invited. According to the present invention, projection data items produced by scanning the next scanned position in the z-axis direction are used to compensate for degradation in image quality.

Referring back to FIG. 15, at step S93, a plurality of sets of back projection data items D2 provided by X-ray beams having passed through the pixel points g(x,y) are weighted and summated in order to produce back projection data items D2′. For example, a plurality of sets of back projection data items D2_z1 and D2_z2 shown in FIG. 20 are weighted and summated in order to produce back projection data items D2′ shown in FIG. 21.
D2′=k1·D2_{—}z1+k2·D2_{—}z2

In the above formula, k1 and k2 denote weighting coefficients that may be set to certain values for brevity's sake. Preferably, the weighting coefficients are determined based on the position of a pixel point expressed by each back projection data D2, or the geometrical position, direction, or angle (α1 or α2 in FIG. 18) of each X-ray beam having passed through the pixel point. In this case, further improvement in image quality is expected. Incidentally, the sum of the weighting coefficient is a unity, that is, k1+k2=1 is established.

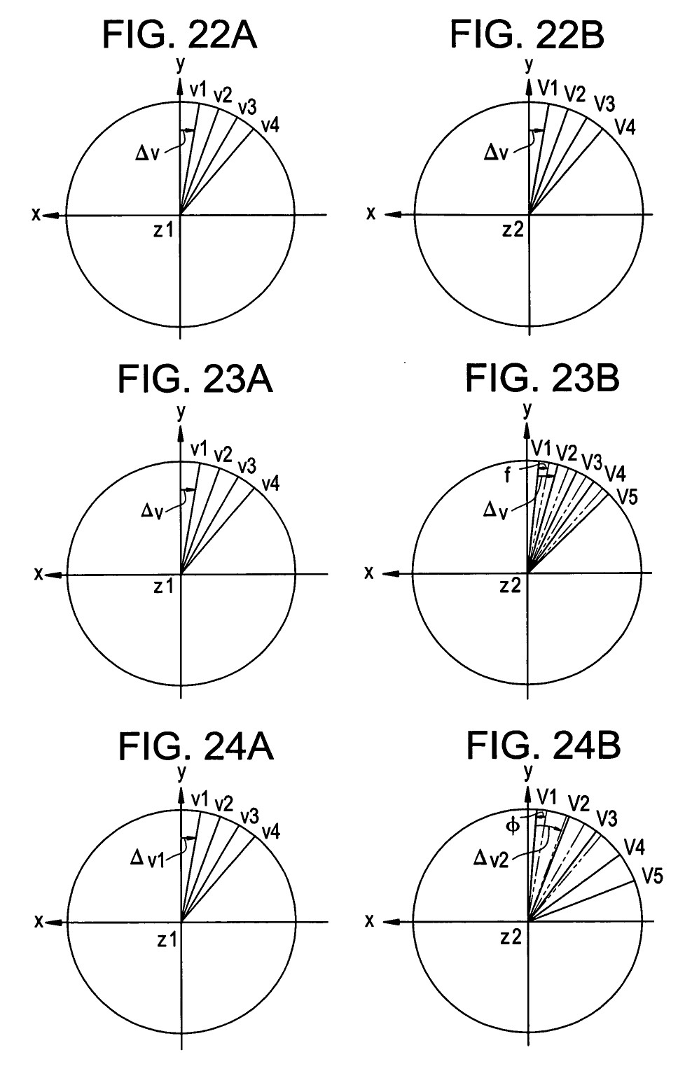

As shown in FIG. 22, when a view angle v1 at which the X-ray tube is disposed for a scan performed on the first scanned position z1 is identical to a view angle V1 at which the X-ray tube is disposed for a scan performed on the second scanned position z2, back projection data items D2(v,x,y)_z1 derived from a view v produced by scanning the first scanned position z1, and back projection data items D2(v,x,y)_z2 derived from the view v produced by scanning the second scanned position z2 are weighted and summated. In this case, since projection data items need not be interpolated using projection data items detected with the X-ray tube disposed at a certain view angle, a reconstructed tomographic image is little blurred.

However, as shown in FIG. 23, when a view angle V1 at which the X-ray tube is disposed for a scan performed on the second scanned position is deviated from a view angle v1, at which the X-ray tube is disposed for a scan performed on the first scanned position z1, by a value −φ(φ<66 v), back projection data items D2(v,x,y)_z1 derived from a view v produced by scanning the first scanned position z1 should be weighted and then summated with back projection data items D2(v,x,y)_z2 that are derived from the view v produced by scanning the second scanned position and that are weighted, and projection data items D2(v+1,x,y)_z2 that are derived from a view v+1 produced by scanning the second scanned position z2 and that are weighted. Thus, projection data items D2 should be produced.

As shown in FIG. 24, assuming the number of views produced during the first scan is different from the number of views produced during the second scan, when a view angle V1 at which the X-ray tube is disposed for the scan performed on the second scanned position z2 is deviated by a value −φ(0≦φ<Δv1) from a view angle v1 at which the X-ray tube is disposed for the scan performed on the first scanned position z1, if a difference between view angles Δv1 at which the X-ray tube is disposed for the scan performed on the first scanned position z1 disagrees from a difference between view angles Δv2 (0<Δv1<Δv2) at which the X-ray tube is disposed for the scan performed on the second scanned position z2, back projection data items D2(V,x,y)_z1 derived from a view V produced by scanning the first scanned position z1 should be weighted and then summated with back projection data items D2(V′,x,y)_z2 that are derived from a view V′ produced by scanning the second scanned position z2 and that are weighted, and back projection data items D2(V′+1,x,y)_z2 that are derived from a view V′+1 produced by scanning the second scanned position z2 and that are weighted. Herein, V′ denotes an integer that meets the condition given by the following formula:
Δv1−φ+(V′−1)×Δv2≦V×Δv1

Otherwise, assuming int{} is a finction that rounds up a real number to provide an integer, V′ is expressed as follows:
V′=int{(V×Δv1−Δv1+φ)/Δv2}

Depending on the positions of pixel points g in a field of view P, one of sets of back projection data items D2_z1 and D2_z2 produced by scanning different scanned positions may lack data. In this case, missing back projection data may be extrapolated in order to produce back projection data items D2′. Otherwise, the cone-beam reconstruction weighting coefficient to be applied to imperfect back projection data items is set to 0 or unused in order to produce the back projection data items D2′ (namely, one of the coefficients k1 and k2 is set to 0 and the other coefficient is set to 1). However, in this case, an X-ray beam and an opposite X-ray beam that pass through the pixel points g are often contained in an X-ray beam CB incident on one scanned position. Therefore, there is no concern about degradation in image quality.

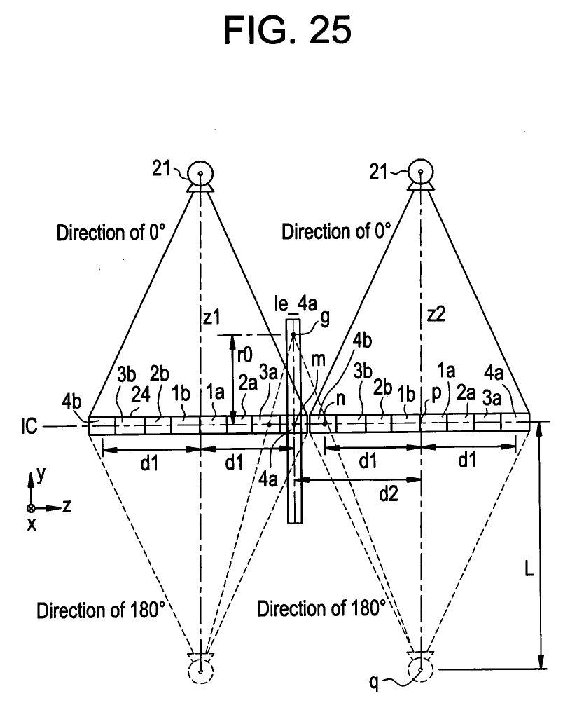

FIG. 25 is an explanatory diagram showing the position of pixel points g in a field of view Ie_4a in a case where back projection data items D2_z1 produced by scanning the first scanned position z1 are available but back projection data items D2_z2 produced by scanning the second scanned position z2 are unavailable.

The field of view Ie_4a shall be associated with a detector array of the multi-array X-ray detector 24 including a detector element 4a during a scan performed on the first scanned position z1. Moreover, d1 denotes a distance from a point on the center axis of rotation aligned with the focal spot in the X-ray tube 21 to the detector element 4a or detector element 4b. Moreover, d2 denotes a distance from the point p on the center axis of rotation Ic aligned with the focal spot in the X-ray tube 21 to the field of view Ie_4a. L denotes a distance from the focal spot in the X-ray tube 21 to the center axis of rotation IC. The d1, d2, and L values are geometrically calculated in relation to the structure of the scanner gantry 20 and stored in the storage device 7.

The length of a side nm is provided as “d2−d1.” Since a triangle gmn and a triangle nqp are similar figures, the length r0 of a side gm is provided as follows:
r0=L(d2−d1)/d1

Assuming that r denotes a distance from the center axis of rotation IC to a pixel point in the field of view Ie_4a, when the distance r to the pixel point is smaller than the length r0, back projection data D2_z2 that is produced by scanning the second scanned position z2 and that represents the pixel point may be unavailable.

When the distance r to a pixel point is larger than the length r0, the coefficients k1 and k2 should preferably be varied depending on the distance r for the purpose of improvement of image quality.

Since the distance r0 is a function of the distances d1 and d2, the coefficients k1 and k2 should preferably be varied depending on the position in the z-axis direction of the field of view for the purpose of improvement of image quality.

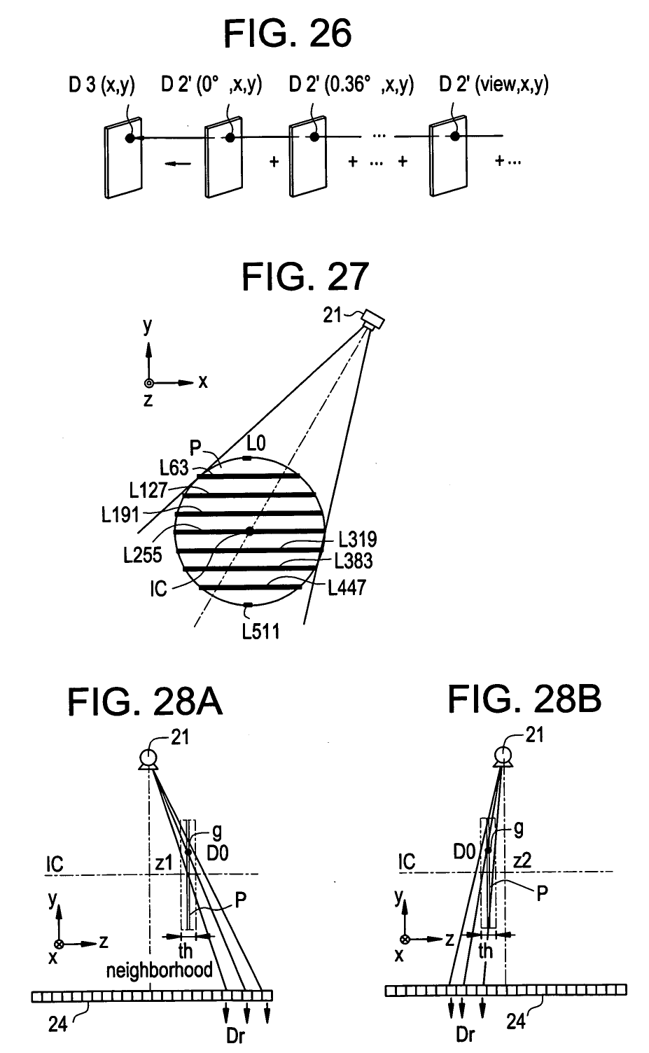

Referring back to FIG. 15, at step S94, as shown in FIG. 26, projection data items D2′ (view,x,y) are added pixel by pixel to back projection data items D3(x,y,z) that are cleared in advance.

At step S95, steps S91 to S94 are repeated for all views needed to reconstruct a tomographic image (namely, views produced by rotating the X-ray tube 360° or 180° +the angle of a fan beam) in order to produce sets of back projection data items D3(x,y,z), that is, tomographic image data items.

The X-ray CT apparatus 100 in accordance with the first embodiment provides the advantages described below.

(1) Sets of projection data items produced by scanning different scanned positions in the z-axis direction are used to reconstruct a tomographic image. Consequently, a high-quality tomographic image little affected by artifacts can be produced.

(2) Sets of projection data items produced by scanning different scanned positions in the z-axis direction are weighted and summated. Therefore, image reconstruction should be performed only once.

A three-dimensional image reconstruction method based on the known Feldkamp technique may be adopted. Alternatively, any of three-dimensional image reconstruction methods proposed in Japanese Unexamined Patent Application Publication Nos. 2003-334188, 2004-41675, 2004-41674, 2004-73360, 2003-159244, and 2004-41675 will do.

Moreover, as shown in FIG. 27, a circular field of view P may be adopted.

Moreover, as shown in FIG. 28, a plurality of projection data items Dr produced by scanning the same scanned position and different scanned positions using X-ray beams that have passed through the same pixel point in the field of view P and a neighborhood th in the z-axis direction centered on the pixel point g may be weighted and summated in order to produce projection data D0.

According to the first embodiment, a z-direction (direction-of-arrays) filter having different coefficients applied to respective detector arrays is used to adjust a difference in image quality derived from a difference in the angle of an X-ray cone beam. Thus, a uniform slice thickness and a uniform image quality determined with artifacts or noises are realized over sets of image data items detected by respective detector arrays. Other various z-direction filters may be used to provide the same advantage.

According to the first embodiment, the spacing W between the first and second scanned positions is set to the width D. As long as the spacing W is equal to or smaller than the width D, image quality can be improved (however, a scanned range is narrowed).

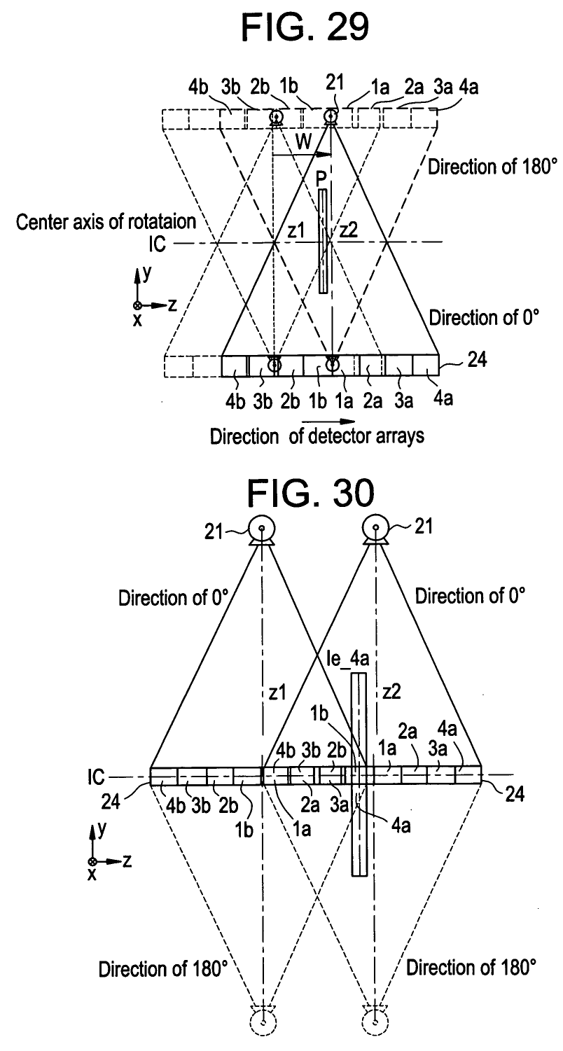

FIG. 29 and FIG. 30 show examples in which W=D/2 is established.

Moreover, the present invention can be adapted to an X-ray CT apparatus including an X-ray area detector represented by a flat-panel detector instead of the multi-array X-ray detector 24.

[Second Embodiment]

According to the first embodiment, after z-filter convolution is completed (step S7 in FIG. 4), sets of projection data items produced by scanning different scanned positions are weighted and summated (step S93 in FIG. 15). According to the second embodiment, when the z-filter convolution is performed, the sets of projection data items produced by scanning different scanned positions are weighted and summated.

Specifically, during z-filter convolution (step S7 in FIG. 4), projection data items provided by an X-ray beam and projection data items provided by an opposite X-ray beam are selected from sets of projection data items produced by scanning the same scanned position and sets of projection data items produced by scanning a different scanned position. A direction-of-arrays filter is applied to the selected sets of projection data items in consideration of a position in the z-axis direction of an X-ray beam providing each set of projection data items.

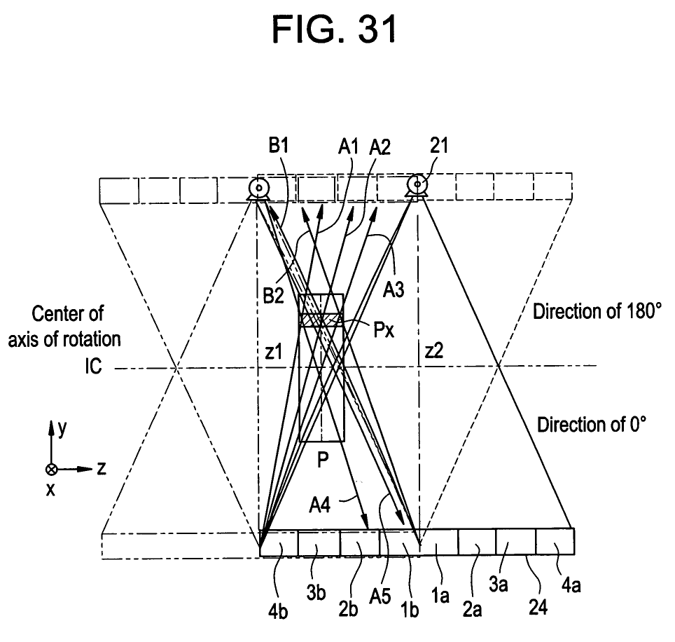

For example, in FIG. 31, when an image of a certain pixel point Px is reconstructed, the direction-of-arrays filter is applied to both projection data items provided by X-ray beams A1 to A5, which have passed through the pixel point Px, during a scan performed on the first scanned position z1, and projection data items provided by X-ray beams B1 and B2, which have passed through the pixel point Px, during a scan performed on the second scanned position z2. At this time, the z-coordinates indicating the first and second scanned positions should be measured or highly precisely inferred in order to accurately select X-ray beams having passed through the pixel point Px. This is important in improvement of image quality through reduction of artifacts.

The second embodiment provides the same advantages as the first embodiment does.

[Third Embodiment]

According to the third embodiment, sets of projection data items produced by scanning different scanned positions in the z-axis direction are used to reconstruct respective tomographic images. The tomographic images are weighted and summated in order to improve image quality.

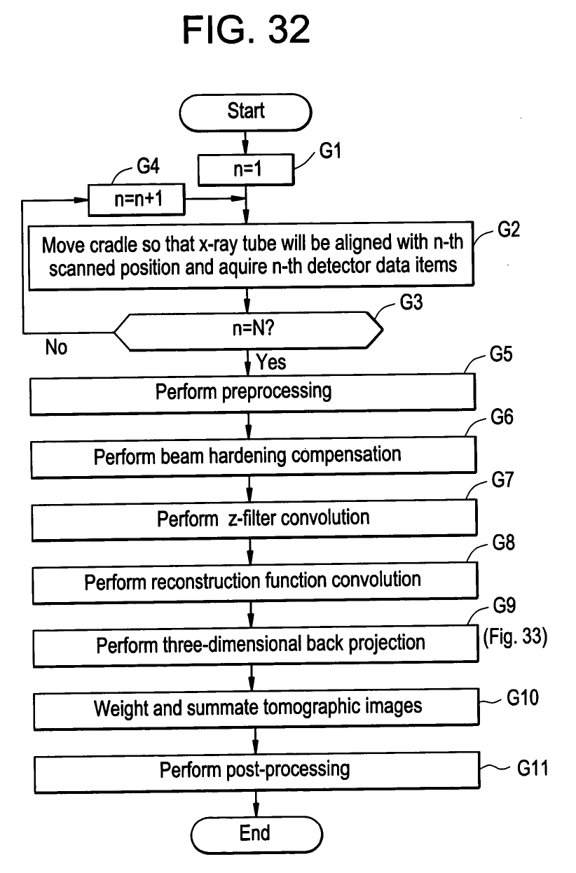

FIG. 32 is a flowchart outlining actions to be performed in an X-ray CT apparatus.

Step G1 to G8 are identical to steps S1 to S8 described in FIG. 4.

At step G9, three-dimensional back projection is performed on projection data items Dr(view,j,i) produced by scanning each scanned position zn. Thus, back projection data items D3(x,y,z), that is, tomographic image data is produced in association with each scanned position zn.

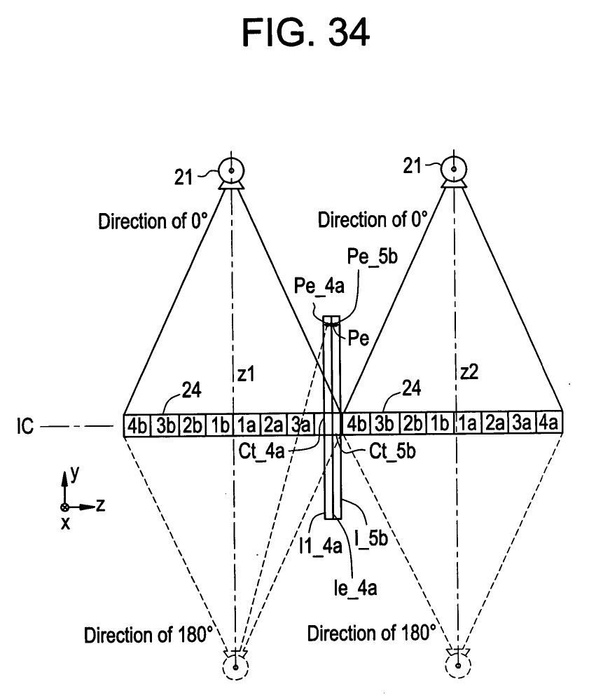

For example, as shown in FIG. 34, a first tomographic image I1_4a is reconstructed based on projection data items produced by scanning the first scanned position z1. A second tomographic image I_5b is reconstructed based on projection data items produced by scanning the second scanned position z2. Incidentally, the tomographic images I1_4a and I_5b express a plane parallel to the xy plane and located at the same position in the z-axis direction. Even when the reconstructed tomographic images express slightly different positions in the z-axis direction, the same advantage is provided. A pixel Pe_4a contained in the tomographic image I1_4a and a pixel Pe_5b contained in the tomographic image I_5b represent the same pixel point indicted with coordinates (x,y).

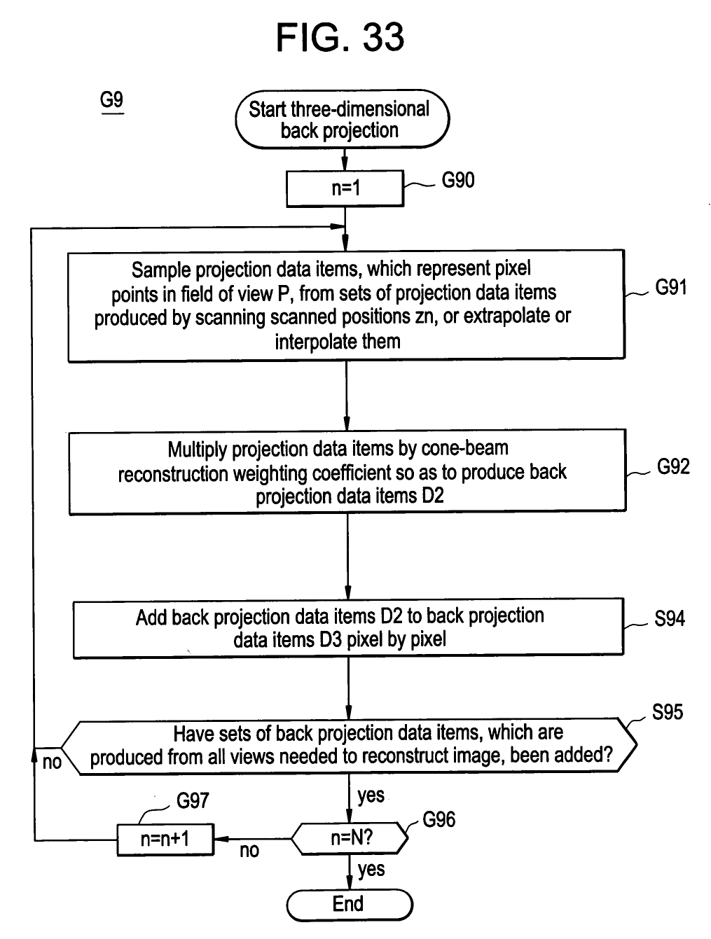

The three-dimensional back projection will be described later with reference to FIG. 33.

At step G10, sets of back projection data items D3(x,y,z) produced by scanning respective scanned positions zn, that is, tomographic image data items are weighted and summated in order to reconstruct a new tomographic image.

For example, the tomographic image I1_4a and tomographic image I_5b shown in FIG. 34 are weighted and summated in order to produce a final tomographic image Ie_4a. Incidentally, the tomographic images I1_4a, Ie_4a, and I_5b express a plane parallel to the xy plane and located at the same position in the z-axis direction. Otherwise, the tomographic images I1_4a and I_5b may express slightly different positions in the z-axis direction, and the tomographic image Ie_4a may express an intermediate position. Even in this case, the same advantage is provided. A pixel Pe_4a contained in the tomographic image I1_4a, a pixel Pe contained in the tomographic image Ie_4a, and a pixel Pe_5b contained in the tomographic image I_5b express the same pixel point indicated with coordinates (x,y).

At step G11, post-processing including image filer convolution and CT number transform is performed on a new tomographic image in order to produce a displayable tomographic image.

The resultant tomographic image is displayed on the display device 6.

FIG. 33 is a flowchart describing three-dimensional back projection (step G9 in FIG. 32).

At step G90, a scanned position counter n is initialized to 1.

Steps G91 and G92 are identical to steps S91 and S92 described in FIG. 15.

At step G94, projection data items D2(view,x,y) are added pixel by pixel to back projection data items D3(x,y,z) that are cleared in advance.

At step S95, steps S91 to S94 are repeated for all views needed to reconstruct a tomographic image (namely, views produced by rotating the X-ray tube 360° or 180°+the angle of a fan beam) in order to produce sets of back projection data items D3(x,y,z), that is, tomographic image data items.

At steps G96 and G97, steps G91 to G96 are repeated until the scanned position counter n indicates a value N. Consequently, sets of back projection data items D3(x,y,z), that is, tomographic image data items expressing each scanned position zn are produced.

Referring to FIG. 34, reconstruction of a tomographic image I1_4a based on projection data items produced by scanning the first scanned position z1 will be discussed below.

Assume that projection data items produced with the X-ray tube disposed at a certain view angle are available but projection data items produced with the X-ray tube disposed at an opposite view angle are unavailable. In this case, the available projection data items are used to reconstruct a tomographic image. For example, an image of a pixel point Pe_4a shown in FIG. 34 is reconstructed based on projection data detected by a detector element 3a with the X-ray tube disposed at a view angle 180°. On the other hand, when both projection data items produced with the X-ray tube disposed at a certain view angle and projection data items produced with the X-ray tube disposed at an opposite view angle are available, both the sets of projection data items are used to reconstruct an image. For example, an image of a center pixel point Ct_4a shown in FIG. 34 is reconstructed based on both projection data detected by a detector element 4a with the X-ray tube disposed at a view angle 0° and projection data detected thereby with the X-ray tube disposed at the view angle 180°.

Next, referring to FIG. 34, reconstruction of a tomographic image I_5b based on projection data items produced by scanning the second scanned position z2 will be discussed below.

When projection data items produced by scanning the second scanned position z2 are available, an image of, for example, a pixel point Pe_5b is reconstructed in the same manner as it is reconstructed using projection data produced by scanning the first scanned position z1. However, projection data items produced by scanning the second scanned position z2 may be totally unavailable. For example, projection data that is produced to represent a center pixel point Ct_5b shown in FIG. 34 by scanning the scanned position z2 is totally unavailable. In this case, a virtual detector element 5b is supposedly located outside the detector element 4b, and projection data items to be produced by a detector array including the detector element 5b are extrapolated for image reconstruction. Otherwise, the weighting coefficient to be applied to projection data items produced by scanning the scanned position z2 is set to 0 so that the projection data items produced by scanning the scanned position z2 will be left unused.

An X-ray CT apparatus in accordance with the third embodiment will provided the advantages described below.

(1) Sets of projection data items produced by scanning different scanned positions in the z-axis direction are used to reconstruct a final tomographic image. Consequently, a high-quality tomographic image little affected by artifacts is produced.

(2) Sets of projection data items produced by scanning different scanned positions in the z-axis direction are used to reconstruct respective tomographic images. Thus, different tomographic images can be produced.

Even when a distance W between the first and second scanned positions z1 and z2 are, as shown in FIG. 29 and FIG. 30, shorter than the width D, the same advantages are provided.

[Fourth Embodiment]

According to the fourth embodiment, tomographic images expressing a plurality of fields of view juxtaposed in the z-axis direction are reconstructed based on projection data items produced by scanning different scanned positions in the z-axis direction. The tomographic images are weighted and summated in order to improve image quality.

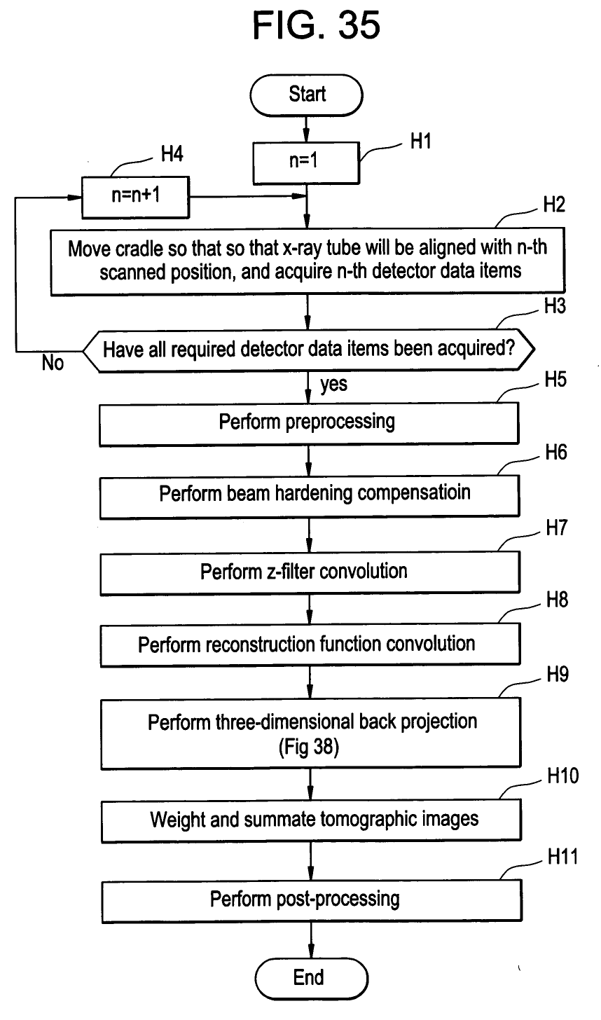

FIG. 35 is a flowchart outlining actions to be performed in an X-ray CT apparatus.

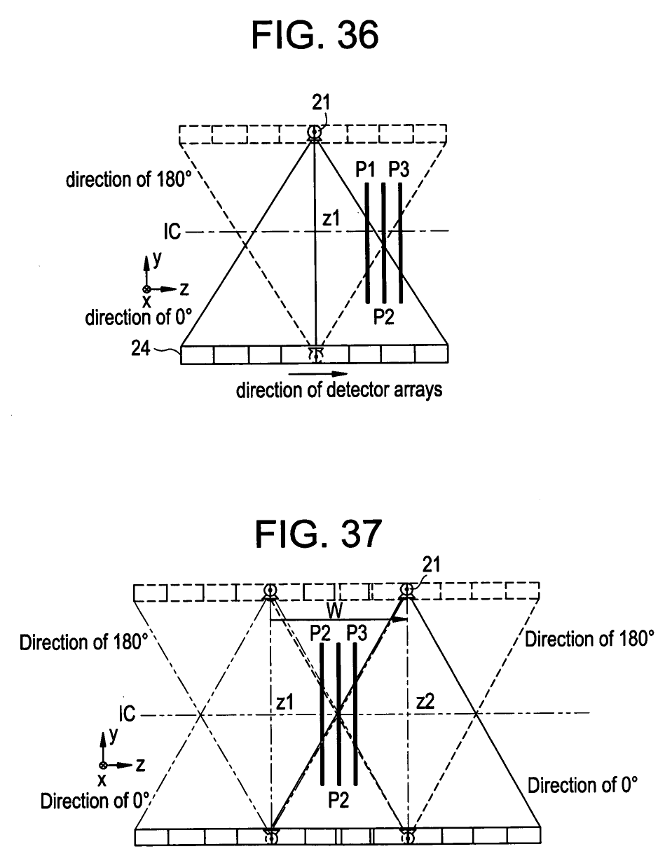

The positions of fields of view P1 to P3 and the first scanned position z1 to N-th scanned positions zN that are shown in FIG. 36 are designated in advance.

At step H1, the scanned position counter n is initialized to 1.

At step H2, the cradle is moved so that the X-ray tube will be aligned with the n-th scanned position zn in the z-axis direction, and the X-ray tube 21 and multi-array X-ray detector 24 are rotated about the center axis of rotation IC. A conventional (axial) scan or cine scan is performed with the cradle 12 left immobilized. Consequently, the n-th X-ray detector data items to which z-axis direction positional information is appended are acquired.

Incidentally, the distance W between scanned positions is larger than 0 and equal to or smaller than the value D, that is, 0<W≦D is established.

At steps H3 and H4, the step H2 is repeated until X-ray detector data items needed to reconstruct tomographic images expressing the pre-designated fields of view P1 to P3 have been acquired.

For example, when a scan is performed on the first scanned position z1, X-ray detector data items representing pixel points located in the center part of the field of view P1 shown in FIG. 36 are acquired with the X-ray tube disposed at both view angles of 0° and 180°. X-ray detector data items representing pixel points located in the perimetric part of the field of view P1 are acquired with the X-ray tube disposed at one of the view angles 0° and 180°. Thus, X-ray detector data items needed to reconstruct an image are acquired by scanning the first scanned position z1. As for the field of view P2, when a scan is performed on the first scanned position z1, X-ray detector data items representing any pixel points are acquired with the X-ray tube disposed at one of the view angles 0° and 180°. Thus, X-ray detector data items needed to reconstruct an image are acquired by scanning the first scanned position z1. As for the perimetric part of the field of view P3, when a scan is performed on the first scanned position z1, X-ray detector data items representing the perimetric part are acquired with the X-ray tube disposed at one of the view angles 0° and 180°. However, X-ray detector data items representing the center part of the field of view P3 are not acquired with the X-ray tube disposed at both the view angles 0° and 180°. Consequently, as shown in FIG. 37, the cradle is moved so that the X-ray tube will be aligned with the second scanned position z2 in order to acquire the second X-ray detector data items. When a scan is performed on the second scanned position z2, X-ray detector data items representing the pixel points in the center part of the field of view P3 are acquired with the X-ray tube disposed at both the view angles 0° and 180°. X-ray detector data items representing the pixel points in the perimetric part of the field of view P3 are acquired with the X-ray tube disposed at the view angle 0° or 180°. Thus, when the X-ray detector data items acquired by scanning both the first and second scanned positions are combined, the X-ray detector data items needed to reconstruct an image are regarded to be acquired.

Referring back to FIG. 35, steps H5 to H8 are identical to steps S1 to S8 described in FIG. 4.

At step H9, three-dimensional back projection is performed on projection data items representing each field of view in order to produce back projection data items, that is, tomographic image data representing each field of view. The three-dimensional back projection will be described with reference to FIG. 39.

At step H10, tomographic images expressing respective fields of view are weighted and summated in order to produce a new tomographic image. For example, assuming that D3(x,y,z)_1, D3(x,y,z)_2, and D3(x,y,z)_3 denote tomographic images expressing the fields of view P1, P2, and P3, D3(x,y,z)_0 denotes a new tomographic image, and w1, w2, and w3 denote weighting coefficients, the new tomographic image D3(x,y,z)_0 is expressed by the following formula:
D3(x,y,z)_{—}0=w1·D3(x,y,z)_{—}1+w2·D3(x,y,z)_{—}2+w3·D3(x,y,z)_{—}3

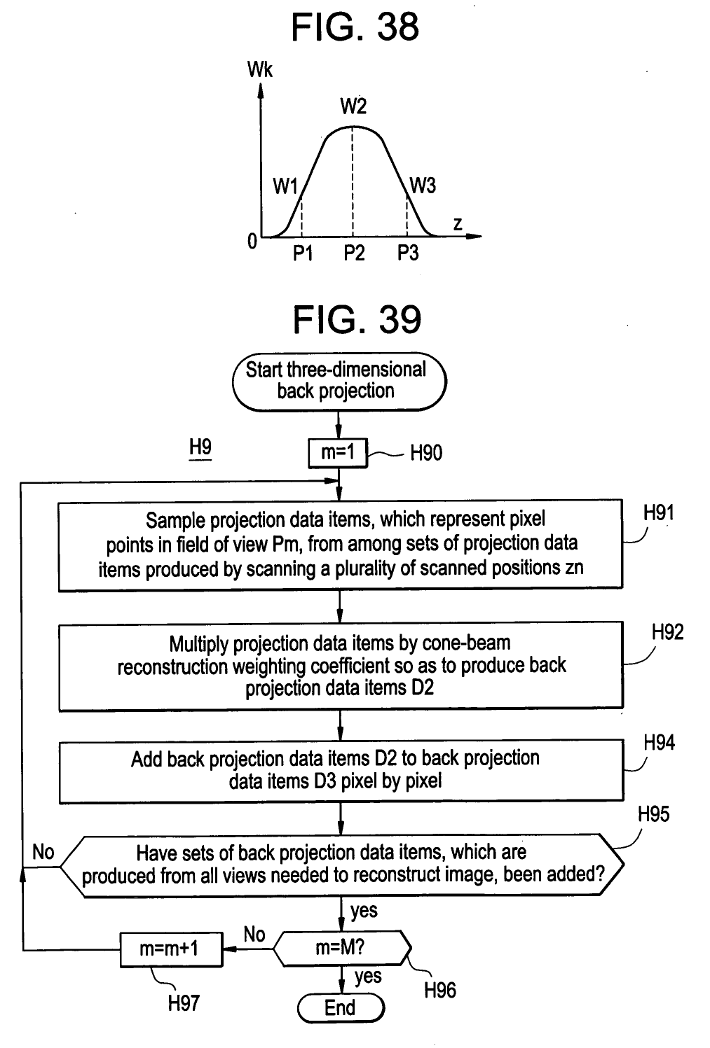

As the weighting coefficients w1, w2, and w3, for example, the coefficients shown in FIG. 38 are adopted. Alternatively, the same coefficients as those defined by the direction-of-arrays filter shown in FIG. 12 or FIG. 14 may be employed.

At step H11, post-processing including image filter convolution and CT number transform is performed on the new tomographic image in order to produce a displayable tomographic image.

The tomographic image is displayed on the display device 6.

FIG. 39 is a flowchart describing three-dimensional back projection (step H9 in FIG. 35).

At step H90, a field-of-view counter m is initialized to 1.

At step H91, one of all views needed to reconstruct a tomographic image is focused. Projection data items representing pixel points in a field of view Pm and being included in the focused view are sampled from sets of projection data items including those produced by scanning a different scanned position zn.

At step H92, the projection data items are multiplied by the cone-beam reconstruction weighting coefficient in order to produce back projection data items D2(view,x,y).

At step H94, the projection data items D2(view,x,y) are added pixel by pixel to back projection data items D3(x,y,z) that are cleared in advance.

At step H95, steps H91 to H94 are repeated for all views needed to reconstruct a tomographic image in order to produce sets of back projection data items D3(x,y,z) representing the pixel points in the field of view Pm, that is, tomographic image data items.

At steps H96 and H97, steps H91 to H95 are repeated until the field-of-view counter m indicates a value M (M denotes 3 in FIG. 37). Consequently, tomographic images expressing the respective fields of view Pm are produced.

The fourth embodiment provides the advantages described below.

(1) After a plurality of tomographic images is reconstructed using sets of projection data items produced by scanning a plurality of different scanned positions in the z-axis direction, the tomographic images are weighted and summated in order to reconstruct a final tomographic image. Consequently, a high-quality tomographic image little affected by artifacts is produced.

(2) Tomographic images expressing a plurality of different fields of view juxtaposed in the z-axis direction are produced. Since an event that any of projection data items needed to reconstruct a tomographic image is missing does not occur, a tomographic image whose quality is more uniform in the z-axis direction can be produced. Thus, image quality improves.

[Fifth Embodiment]

In the aforesaid embodiments, the cradle 12 is moved in the z-axis direction in order to perform a conventional (axial) scan or a cine scan. Alternatively, the scanner gantry 20 may be moved in the z-axis direction in order to perform a conventional (axial) scan or a cine scan. In short, the rotator and a subject should merely be relatively moved in the z-axis direction.

[Sixth Embodiment]

Radiation employed for scanning is not limited to X-rays but may be gamma rays or any other radiation.

Many widely different embodiments of the invention may be configured without departing from the spirit and the scope of the present invention. It should be understood that the present invention is not limited to the specific embodiments described in the specification, except as defined in the appended claims.

1. An X-ray CT apparatus comprising: a scan device that rotates an X-ray generator and an X-ray area detector which is opposed to the X-ray generator, is represented by an X-ray area detector represented by a multi-array X-ray detector or a flat-panel detector, and has a matrix structure, with a center axis of rotation, which is located between the X-ray generator and X-ray area detector, as a center so as to detect projection data items of a subject lying down between the X-ray generator and X-ray area detector; a z-coordinate positional information acquisition device for acquiring z-coordinate positional information concerning a position, at which each set of projection data items is detected, on the assumption that the direction of the center axis of rotation corresponds to the direction of a z axis; a three-dimensional image reconstruction device for reconstructing a three-dimensional tomographic image on the basis of the detected projection data items in consideration of the z-coordinate positional information; and a tomographic image display device for displaying the tomographic image, the X-ray CT apparatus further comprising:
a first scan device for detecting the first projection data items by performing the first conventional (axial) scan or cine scan on the first scanned position in the z-axis direction;
a second scan device for producing the second projection data items by performing the second conventional scan or cine scan on the second scanned position in the z-axis direction at which the range in the z-axis direction of an X-ray beam substantially communicates with or overlaps the range in the z-axis direction of an X-ray beam incident on the first scanned position; and
a tomographic image reconstruction device for reconstructing a tomographic image, which expresses a position on the subject falling within a range from the z-coordinate position at which the first projection data items are detected to the z-coordinate position at which the second projection date items are detected, by utilizing both the first projection data items and the second projection data items.

2. The radiation CT method according to claim 1, wherein the tomographic image reconstruction device selects projection data items, which are provided by an X-ray beam having passed through pixel points in a subject's section, from each of the first projection data items and the second projection data items, weights and summates the sets of projection data items selected from the first projection data items and the second projection data items respectively, and uses the summated projection data items to produce a three-dimensional tomographic image.

3. An X-ray CT apparatus comprising: a scan device that rotates an X-ray generator and an X-ray area detector which is opposed to the X-ray generator, is represented by an X-ray area detector represented by a multi-array X-ray detector or a flat-panel detector, and has a matrix structure, with a center axis of rotation, which is located between the X-ray generator and X-ray area detector, as a center so as to detect projection data items of a subject lying down between the X-ray generator and X-ray area detector; a z-coordinate positional information acquisition device for acquiring z-coordinate positional information concerning a position, at which each set of projection data items is detected, on the assumption that the direction of the center axis of rotation corresponds to the direction of a z axis; a three-dimensional image reconstruction device for reconstructing a three-dimensional tomographic image on the basis of the detected projection data items in consideration of the z-coordinate positional information; and a tomographic image display device for displaying the tomographic image, the X-ray CT apparatus further comprising:
a first scan device for detecting the first projection data items by performing the first conventional (axial) scan or cine scan on the first scanned position in the z-axis direction;
a second scan device for detecting the second projection data items by performing the second conventional scan or cine scan on the second scanned position in the z-axis direction at which the range in the z-axis direction of an X-ray beam substantially communicates with or overlaps the range in the z-axis direction of an X-ray beam incident on the first scanned position; and
a tomographic image reconstruction device for selecting projection data items, which are provided by an X-ray beam having passed through pixel points in a subject's section, from each of the first projection data items and the second projection data items, weighting and summating the sets of projection data items selected from the first projection data items and the second projection data items respectively, and reconstructing a three-dimensional tomographic image on the basis of at least one of the projection data items selected from the first projection data items, the projection data items selected from the second projection data items, and the summated projection data items.

4. An X-ray CT apparatus comprising: a scan device that rotates an X-ray generator and an X-ray area detector, which is opposed to the X-ray generator, is represented by an X-ray area detector represented by a multi-array X-ray detector or a flat-panel detector, and has a matrix structure, with a center axis of rotation, which is located between the X-ray generator and X-ray area detector, as a center so as to detect projection data items of a subject lying down between the X-ray generator and X-ray area detector; a z-coordinate positional information acquisition device for acquiring z-coordinate positional information concerning a position, at which each set of projection data items is detected, on the assumption that the direction of the center axis of rotation corresponds to the direction of a z axis; a three-dimensional image reconstruction device for reconstructing a three-dimensional tomographic image on the basis of the detected projection data items in consideration of the z-coordinate positional information; and a tomographic image display device for displaying the tomographic image, the X-ray CT apparatus further comprising:
an n-th scan device for detecting the n-th projection data items by performing the n-th conventional (axial) scan or cine scan on the n-th scanned position (where n denotes an integer ranging from 1 to N where N denotes an integer equal to or larger than 2) in the z-axis direction at which the range in the z-axis direction of an X-ray beam substantially communicates with or overlaps the range in the z-axis direction of an X-ray beam incident on an adjoining scanned position; and
a tomographic image reconstruction device for reconstructing a tomographic image, which expresses a position on the subject falling within a range from the z-coordinate position at which the first projection data items are detected to the z-coordinate position at which the N-th projection data items are detected, by utilizing one or a plurality of the first to N-th sets of projection data items.

5. The X-ray CT apparatus according to claim 1, wherein the spacing between adjoining scanned positions is substantially equal to or smaller than the width D of an X-ray cone beam on the center axis of rotation.

6. The X-ray CT apparatus according to claim 1, wherein when a plurality of sets of projection data items is utilized, the tomographic image reconstruction device uses coefficients, which depend on the geometric positions and directions of X-ray beams providing the respective sets of projection data items, to weight the sets of projection data items, summates the resultant sets of projection data items, and reconstructs a three-dimensional tomographic image on the basis of at least one of the projection data items selected from the n-th projection data items and the summated projection data items.

7. The X-ray CT apparatus according to claim 1, wherein when the scan device detects projection data items, the X-ray generator is disposed at the same view angle for the respective scans performed on adjoining scanned positions.

8. The X-ray CT apparatus according to claim 1, wherein: when the scan device detects projection data items, the X-ray generator is not necessarily disposed at the same view angle for the respective scans performed on adjoining scanned positions; and when a plurality of sets of projection data items is utilized, if the tomographic image reconstruction device cannot sample sets of projection data items that are detected during the respective scans performed on different scanned positions with the X-ray generator disposed at the same view angle, the tomographic image reconstruction device weights and summates the sets of projection data items in consideration of the view angles at which the X-ray generator is disposed in order to detect the sets of projection data items.

9. The X-ray CT apparatus according to claim 1, wherein: when the scan device detects projection data items, the X-ray generator is not necessarily disposed at the same view angle for the respective scans performed on adjoining scanned positions; and when a plurality of sets of projection data items is utilized, if the tomographic image reconstruction device cannot sample projection data items that are detected during the respective scans performed on different scanned positions with the X-ray generator disposed at the same view angle, the tomographic image reconstruction device synthesizes sets of projection data items detected during one scan performed on one scanned position with the X-ray generator disposed at different view angles so as to detect projection data items that are regarded as projection data items produced with the X-ray generator disposed at the same view angle.

10. The X-ray CT apparatus according to claim 1, wherein the tomographic image reconstruction device selects projection data items, which are provided by an X-ray beam having passed through pixel points in a subject's section, from sets of projection data items detected by scanning respective scanned positions, uses the sets of projection data items, which are selected from the sets of projection data items detected by scanning the respective scanned positions, to reconstruct three-dimensional images expressing the respective scanned positions, and weights and summates the resultant tomographic images expressing the respective scanned positions so as to reconstruct a tomographic image.

11. The X-ray CT apparatus according to claim 10, wherein coefficients dependent on the geometric conditions including scanned positions corresponding to subject's sections, the positions in the z-axis direction of the sections, the slice thicknesses of the sections, the positions of pixel points in each of the sections, the position and size of the focal spot in the X-ray generator, and the position and size of the X-ray area detector are used to weight tomographic images expressing the respective scanned positions, and the resultant tomographic images are summated.

12. The X-ray CT apparatus according to claim 4, wherein when a plurality of sets of projection data items is utilized, the tomographic image reconstruction device selects projection data items, which are provided by an X-ray beam having passed though pixel points in a subject's section, from sets of projection data items detected by scanning respective scanned positions, uses the sets of projection data items, which are selected from the sets of projection data items detected by scanning the respective scanned positions, to reconstruct three-dimensional images expressing the respective scanned positions, and weights and summates the resultant tomographic images expressing the respective scanned positions so as to reconstruct a tomographic image.

13. The X-ray CT apparatus according to claim 3, wherein the spacing between adjoining scanned positions is substantially equal to or smaller than the width D of an X-ray cone beam on the center axis of rotation.

14. The X-ray CT apparatus according to claim 3, wherein when a plurality of sets of projection data items is utilized, the tomographic image reconstruction device uses coefficients, which depend on the geometric positions and directions of X-ray beams providing the respective sets of projection data items, to weight the sets of projection data items, summates the resultant sets of projection data items, and reconstructs a three-dimensional tomographic image on the basis of at least one of the projection data items selected from the n-th projection data items and the summated projection data items.

15. The X-ray CT apparatus according to claim 3, wherein when the scan device detects projection data items, the X-ray generator is disposed at the same view angle for the respective scans performed on adjoining scanned positions.

16. The X-ray CT apparatus according to claim 3, wherein: when the scan device detects projection data items, the X-ray generator is not necessarily disposed at the same view angle for the respective scans performed on adjoining scanned positions; and when a plurality of sets of projection data items is utilized, if the tomographic image reconstruction device cannot sample sets of projection data items that are detected during the respective scans performed on different scanned positions with the X-ray generator disposed at the same view angle, the tomographic image reconstruction device weights and summates the sets of projection data items in consideration of the view angles at which the X-ray generator is disposed in order to detect the sets of projection data items.

17. The X-ray CT apparatus according to claim 3, wherein: when the scan device detects projection data items, the X-ray generator is not necessarily disposed at the same view angle for the respective scans performed on adjoining scanned positions; and when a plurality of sets of projection data items is utilized, if the tomographic image reconstruction device cannot sample projection data items that are detected during the respective scans performed on different scanned positions with the X-ray generator disposed at the same view angle, the tomographic image reconstruction device synthesizes sets of projection data items detected during one scan performed on one scanned position with the X-ray generator disposed at different view angles so as to detect projection data items that are regarded as projection data items produced with the X-ray generator disposed at the same view angle.

18. The X-ray CT apparatus according to claim 4, wherein the spacing between adjoining scanned positions is substantially equal to or smaller than the width D of an X-ray cone beam on the center axis of rotation.

19. The X-ray CT apparatus according to claim 4, wherein when a plurality of sets of projection data items is utilized, the tomographic image reconstruction device uses coefficients, which depend on the geometric positions and directions of X-ray beams providing the respective sets of projection data items, to weight the sets of projection data items, summates the resultant sets of projection data items, and reconstructs a three-dimensional tomographic image on the basis of at least one of the projection data items selected from the n-th projection data items and the summated projection data items.

20. The X-ray CT apparatus according to claim 4, wherein when the scan device detects projection data items, the X-ray generator is disposed at the same view angle for the respective scans performed on adjoining scanned positions.

21. The X-ray CT apparatus according to claim 4, wherein: when the scan device detects projection data items, the X-ray generator is not necessarily disposed at the same view angle for the respective scans performed on adjoining scanned positions; and when a plurality of sets of projection data items is utilized, if the tomographic image reconstruction device cannot sample sets of projection data items that are detected during the respective scans performed on different scanned positions with the X-ray generator disposed at the same view angle, the tomographic image reconstruction device weights and summates the sets of projection data items in consideration of the view angles at which the X-ray generator is disposed in order to detect the sets of projection data items.

22. The X-ray CT apparatus according to claim 4, wherein: when the scan device detects projection data items, the X-ray generator is not necessarily disposed at the same view angle for the respective scans performed on adjoining scanned positions; and when a plurality of sets of projection data items is utilized, if the tomographic image reconstruction device cannot sample projection data items that are detected during the respective scans performed on different scanned positions with the X-ray generator disposed at the same view angle, the tomographic image reconstruction device synthesizes sets of projection data items detected during one scan performed on one scanned position with the X-ray generator disposed at different view angles so as to detect projection data items that are regarded as projection data items produced with the X-ray generator disposed at the same view angle.
